(19) United States
(12) Patent Application Publication
(10) Pub. No.: US 2026/0151123 A1
(43) Pub. Date: Jun. 4, 2026

(54) A DEVICE FOR TREATING A FISTULA

(71) Applicant: UNIVERSITY HOSPITALS BIRMINGHAM NHS FOUNDATION TRUST, Edgbaston, Birmingham (GB)

(72) Inventors: Timothy George HENSON, Sutton Coldfield (GB); Michael Robert Burch KEIGHLEY, Tanworth-in-Arden, Solihull (GB); Elizabeth LI, Birmingham (GB); Thomas PINKNEY, Tanworth-in-Arden, Solihull (GB); Michael John RAXWORTHY, York (GB)

(73) Assignee: UNIVERSITY HOSPITALS BIRMINGHAM NHS FOUNDATION TRUST, Edgbaston, Birmingham (GB)

(21) Appl. No.: 19/122,389

(22) PCT Filed: Oct. 20, 2023
(86) PCT No.: PCT/GB2023/052738

§ 371 Date: Apr. 18, 2025

(30) Foreign Application Priority Data
Oct. 20, 2022 (GB) 2215523.8

(51) Int. Cl. A61B 17/00

(52) U.S. Cl. CPC A61B 17/0057; A61B 2017/00004; A61B 2017/00526; A61B 2017/00641

(57) ABSTRACT

The present invention relates to devices and related methods for treating fistulas such as anal or recto-vaginal fistulas. Typically, the devices comprise a seton and an elongate body of tissue growth promoting matrix material, wherein the elongate body is suitable for being positioned within the fistula tract. In one aspect of the invention the elongate body comprises a plurality of protrusions positioned on at least one outer surface of said elongate body. In another aspect of the invention, the device comprises one or more attachment points positioned along the length of the elongate body, wherein the one or more attachment points are configured to connect the elongate body to a further seton.

### FIELD OF THE INVENTION

The present invention relates to devices and related methods for treating fistulas such as anal or recto-vaginal fistulas. Typically, the devices comprise a seton and an elongate body of tissue growth promoting matrix material, wherein the elongate body is suitable for being positioned within the fistula tract. In one aspect of the invention the elongate body comprises a plurality of protrusions positioned on at least one outer surface of said elongate body. In another aspect of the invention, the device comprises one or more attachment points positioned along the length of the elongate body, wherein the one or more attachment points are configured to connect the elongate body to a further seton.

### BACKGROUND OF THE INVENTION

An anal fistula, otherwise known as an anorectal fistula, is an abnormal passage formed between the wall of the anal canal and the skin around the anus, typically the perianal skin. An anal fistula usually originates from an infection in an anal gland located in the anal canal. In the case of an anal gland becoming infected, an abscess may form deep under the skin around the anus which requires surgical drainage. After drainage, a tract between the drainage site and the wall of the anal canal may form resulting in an anal fistula. Fistulas cause intermittent symptoms of discharge and generally do not heal without treatment or surgical intervention. Anal fistulas are also a common feature of inflammatory bowel diseases, especially ulcerative colitis and Crohn's disease.

‘Lay open’ fistulotomy is the conventional surgery for treating an anal fistula and involves dividing the tissue between the fistula and the skin so as to promote tissue regeneration and hence healing of the fistula. A disadvantage with this procedure is that it causes discomfort and scarring, and usually results in some level of incontinence.

In an alternative procedure, a seton may be used which is passed through the track of the fistula by use of a fistula probe. The seton is a string preferably formed out of silicon or rubber that is typically threaded through an eye of the fistula probe. The probe is then passed through the track of the fistula pulling the seton along so that it extends through the entire length of the fistula. As the probe reaches the wall of the anal canal, the probe is passed through the anus and then removed from the seton such that the two loose ends of the seton can be tied together so as to form a loop, thus forming a ‘seton stitch’. The seton stitch is typically either left in place long-term and assists in draining any discharge from the fistula, or is tied tight to produce a slow form of fistulotomy, that is, division of tissues superficial to the fistula.

Where the seton stitch is left in place long-term to assist in draining any discharge from the fistula, the results can be variable. In particular, where the fistula is narrow and/or the discharge is viscous, the drainage is often inadequate, prolonging and/or worsening any infection. Persistent infection makes definitive treatment difficult and less successful.

An entirely new procedure for the treatment of fistulas is described in patent application WO 2011/151659. The procedure uses a seton stitch to secure a tissue growth promoter, such as a fibrin plug, within a fistula. The procedure thereby allows and encourages the fistula to heal internally, rather than merely drain. Furthermore, the procedure avoids the major drawbacks associated with fistulotomy, as discussed above. Accordingly, it can be seen that the new procedure represents a major advancement in the clinical treatment of fistulas.

Also described in patent application WO 2011/151659 are devices suitable for use in the new procedure. For example, in one embodiment a device comprising a fistula plug secured to a seton is described, wherein the seton is thread through a hole in the centre of the fistula plug. In another embodiment, a fistula plug sutured to a seton is described. Additional devices for use in the new procedure are described in patent application WO 2014/023962.

There is however a need for further devices which better secure the tissue growth promoter within the fistula tract, which allow the end user to more easily treat complex fistulas, and/or which are cost effective to manufacture.

The present invention seeks to provide devices that fulfil some or all of the aforementioned needs.

### SUMMARY OF THE INVENTION

According to a first aspect of the present invention, there is provided a device suitable for treating a fistula, wherein the fistula comprises a fistula tract, wherein the device comprises a seton and an elongate body of tissue growth promoting matrix material, wherein the elongate body is suitable for being positioned within the fistula tract, and wherein the elongate body comprises a plurality of protrusions positioned on at least one outer surface of said elongate body.

In one embodiment of the first aspect of the invention, the elongate body has a longitudinal axis. Typically, one or more of the plurality of protrusions are positioned on an outer surface that is parallel or substantially parallel to the longitudinal axis of the elongate body. More typically, two or more of the plurality of protrusions are positioned on an outer surface that is parallel or substantially parallel to the longitudinal axis of the elongate body.

The plurality of protrusions may be positioned on a single outer surface or on two or more outer surfaces of the elongate body. Typically, each outer surface on which the plurality of protrusions are positioned is parallel or substantially parallel to the longitudinal axis of the elongate body. For example, in one embodiment the elongate body comprises a first outer surface and a second outer surface, wherein the first outer surface and the second outer surface are both parallel or substantially parallel to the longitudinal axis of the elongate body, wherein a first plurality of protrusions is positioned on the first outer surface, and wherein a second plurality of protrusions is positioned on the second outer surface.

In one embodiment, each outer surface on which each plurality of protrusions are positioned comprises from 2 to 200 protrusions. More typically, each outer surface on which the plurality of protrusions are positioned comprises from 5 to 100 protrusions, or from 10 to 60 protrusions. More typically still, each outer surface on which the plurality of protrusions are positioned comprises from 30 to 50 protrusions.

The plurality of protrusions may be distributed uniformly or non-uniformly across each outer surface upon which they are positioned. The plurality of protrusions may be distributed across all or part of each outer surface upon which they are positioned.

Typically, the plurality of protrusions is distributed substantially uniformly across substantially all of each outer surface upon which they are positioned.

In one embodiment the distance (dp) between the maxima of each neighbouring protrusion on each outer surface on which the plurality of protrusions are positioned is between 0.1 and 20 mm. Typically, the distance (dp) between the maxima of each neighbouring protrusion is between 0.2 and 10 mm. More typically, the distance (dp) between the maxima of each neighbouring protrusion is between 0.5 and 5 mm. More typically still, the distance (dp) between the maxima of each neighbouring protrusion is between 1 and 3 mm.

In one embodiment, each protrusion of the plurality of protrusions has a height of between 0.1 and 5 mm. More typically, each protrusion of the plurality of protrusions has a height of between 0.5 and 3 mm. More typically still, each protrusion of the plurality of protrusions has a height of between 0.8 and 1.5 mm. As will be understood, as referred to herein the height of a protrusion refers to the maximum distance (hp) that the protrusion extends from a neighbouring minima of the surface, as measured perpendicular to the surface on which the protrusion is formed.

In one embodiment, each protrusion of the plurality of protrusions has a width of between 0.1 and 10 mm. More typically, each protrusion of the plurality of protrusions has a width of between 0.5 and 5 mm. More typically still, each protrusion of the plurality of protrusions has a width of between 1 and 3 mm. As will be understood, as referred to herein the width of a protrusion refers to the maximum distance (Wp) that the protrusion extends between two neighbouring minima, as measured parallel to the surface on which the protrusion is formed.

Each protrusion of the plurality of protrusions may have the same or different dimensions. Typically, each protrusion of the plurality of protrusions has substantially the same dimensions. More typically, each protrusion of the plurality of protrusions has the same dimensions.

Each protrusion of the plurality of protrusions may have the same or a different shape. For example, each protrusion of the plurality of protrusions may be rounded or domed, cylindrical, conical, squared or cuboid, or triangular, triangular prismatic or pyramidal in shape. Typically, each protrusion comprises at least one point. In one embodiment, each protrusion of the plurality of protrusions is conical, triangular, triangular prismatic or pyramidal in shape. Typically, each protrusion of the plurality of protrusions is substantially the same in shape. More typically, each protrusion of the plurality of protrusions is the same in shape.

In one embodiment, each minima between neighbouring protrusions is not pointed or vee-shaped. For example, each minima between neighbouring protrusions may be curved or rounded, e.g. so as to form a concave curve between the maxima of each neighbouring protrusion.

The elongate body may be cylindrical, conical or prismatic in form. The prismatic form may be regular or irregular. For example, the elongate body may be cylindrical, conical, triangular prismatic, square prismatic, rectangular prismatic, pentagonal prismatic or hexagonal prismatic in form. In such embodiments, one or more of the plurality of protrusions are typically positioned on one or more of the side faces of the cylindrical, conical or prismatic form. Any edges or corners formed between an end face and one or more of the side faces of the cylindrical, conical or prismatic form may optionally be chamfered.

The plurality of protrusions may be formed from the same or a different material to the elongate body of tissue growth promoting matrix material. Typically, the plurality of protrusions are formed from one or more biodegradable materials such as those discussed below. Typically, the plurality of protrusions are formed from the same material as the elongate body.

In one embodiment, the elongate body and the plurality of protrusions are of unitary construction.

The elongate body may be defined such that it has a longitudinal axis (x) and two further axes (y) and (z) that are perpendicular to each other and perpendicular to the longitudinal axis (x). When so defined, the maximum length of the elongate body as measured along the x-axis is greater than both the maximum width and the maximum height of the elongate body as measured along the y- and z-axis respectively.

Typically, the maximum length of the elongate body as measured along the x-axis is from 2 to 30 times greater than the larger of the maximum of the width and the maximum of the height of the elongate body as measured along the y- and z-axis respectively. More typically, the maximum length of the elongate body is from 5 to 15 times greater than the larger of the maximum of the width and the maximum of the height of the elongate body. More typically, the maximum length of the elongate body is from 8 to 12 times greater than the larger of the maximum of the width and the maximum of the height of the elongate body.

In one embodiment, the maximum length of the elongate body as measured along the x-axis is from 10 to 200 mm. Typically, the maximum length of the elongate body is from 30 to 150 mm. More typically, the maximum length of the elongate body is from 50 to 100 mm.

In one embodiment, the larger of the maximum of the width or the maximum of the height of the elongate body, as measured along the y- and z-axis respectively, is from 3 to 20 mm. Typically, the larger of the maximum of the width or the maximum of the height of the elongate body is from 4 to 15 mm. More typically, larger of the maximum of the width or the maximum of the height of the elongate body is from 5 to 10 mm.

In one embodiment, the elongate body is an elongate sheet of tissue growth promoting matrix material. In such an embodiment, the elongate sheet may be defined such that when laid flat it has a longitudinal axis (x) and two further axes (y) and (z) that are perpendicular to each other and perpendicular to the longitudinal axis (x). Typically, when laid flat the y-axis is parallel to the plane of the sheet and the z-axis is perpendicular to the plane of the sheet. When so defined, the maximum length of the elongate sheet as measured along the x-axis is greater than the maximum width of the elongate sheet as measured along the y-axis, and the maximum width of the elongate sheet as measured along the y-axis is greater than the maximum height (i.e. the thickness) of the elongate sheet as measured along the z-axis.

Typically, the maximum length of the elongate sheet as measured along the x-axis is from 2 to 30 times greater than the maximum width of the elongate sheet as measured along the y-axis. More typically, the maximum length of the elongate sheet is from 5 to 15 times greater than the maximum width of the elongate sheet. More typically, the maximum length of the elongate sheet is from 8 to 12 times greater than the maximum width of the elongate sheet.

Typically, the maximum width of the elongate sheet as measured along the y-axis is from 5 to 50 times greater than the maximum height or thickness of the elongate sheet as measured along the z-axis. More typically, the maximum width of the elongate sheet is from 10 to 30 times greater than the maximum height or thickness of the elongate sheet. More typically, the maximum width of the elongate sheet is from 15 to 20 times greater than the maximum height or thickness of the elongate sheet.

In one embodiment, the maximum length of the elongate sheet as measured along the x-axis is from 10 to 200 mm. Typically, the maximum length of the elongate sheet is from 30 to 150 mm. More typically, the maximum length of the elongate sheet is from 50 to 100 mm.

In one embodiment, the maximum width of the elongate sheet as measured along the y-axis is from 3 to 20 mm. Typically, the maximum width of the elongate sheet is from 4 to 15 mm. More typically, the maximum width of the elongate sheet is from 5 to 10 mm. In one embodiment, the maximum height or thickness of the elongate sheet as measured along the z-axis is from 0.1 to 2 mm. Typically, the maximum height or thickness of the elongate sheet is from 0.2 to 1 mm. More typically, the maximum height or thickness of the elongate sheet is from 0.3 to 0.5 mm.

Typically, the elongate sheet of tissue growth promoting matrix material is substantially rectangular, trapezoid or oval in shape. More typically, the elongate sheet of tissue growth promoting matrix material is substantially rectangular or trapezoid in shape.

Most typically, the elongate sheet of tissue growth promoting matrix material is substantially rectangular in shape. Where the elongate sheet is substantially rectangular or trapezoid in shape, the corners of the rectangle or trapezoid may optionally be rounded or chamfered.

Where the elongate body is an elongate sheet of tissue growth promoting matrix material, in one embodiment the plurality of protrusions are positioned on at least one outer edge surface of the elongate sheet. As will be understood, the term ‘outer edge surface’ when used in reference to the elongate sheet refers to an outer surface formed in at least one dimension that is parallel or substantially parallel to the thickness of the sheet (i.e. the z-axis as defined above).

Typically, one or more of the plurality of protrusions are positioned on an outer edge surface that is parallel or substantially parallel to the longitudinal axis of the elongate sheet. In such an embodiment, the outer edge surface can be seen to extend in the direction of the x- and the z-axes, as defined above. More typically, two or more of the plurality of protrusions are positioned on an outer edge surface that is parallel or substantially parallel to the longitudinal axis of the elongate sheet.

The plurality of protrusions may be positioned on a single outer edge surface or on two or more outer edge surfaces of the elongate sheet. Typically, each outer edge surface on which the plurality of protrusions are positioned is parallel or substantially parallel to the longitudinal axis of the elongate body. For example, in one embodiment the elongate sheet comprises a first outer edge surface and a second outer edge surface, wherein the first outer edge surface and the second outer edge surface are both parallel or substantially parallel to the longitudinal axis of the elongate sheet, wherein a first plurality of protrusions is positioned on the first outer edge surface, and wherein a second plurality of protrusions is positioned on the second outer edge surface.

In one embodiment, the elongate sheet does not comprise any protrusions on any surface that is parallel or substantially parallel to both the x- and y-axes, i.e. any surface that is parallel or substantially parallel to the plane of the sheet when the sheet is laid flat.

Where the plurality of protrusions are positioned on at least one outer edge surface of the elongate sheet, the plurality of protrusions may be formed by cutting the outer edge of the elongate sheet so as to form the plurality of protrusions. For example, the elongate sheet may be cut so as to have one or more castellated or serrated outer edges. Typically, the elongate sheet is cut so as to have one or more serrated outer edges.

Accordingly, in an exemplary embodiment of the first aspect of the invention, there is provided a device suitable for treating a fistula, wherein the fistula comprises a fistula tract, wherein the device comprises a seton and an elongate sheet of tissue growth promoting matrix material, wherein the elongate sheet is suitable for being positioned within the fistula tract, and wherein the elongate sheet comprises a first outer edge surface and a second outer edge surface, wherein the first outer edge surface and the second outer edge surface are both parallel or substantially parallel to the longitudinal axis of the elongate sheet when laid flat, wherein a first plurality of protrusions is formed by serrations on the first outer edge surface, and wherein a second plurality of protrusions is formed by serrations on the second outer edge surface.

In accordance with the first aspect of the invention, the seton may be affixed to the elongate body or elongate sheet of tissue growth promoting matrix material.

In one embodiment, the seton is thread through the elongate body or elongate sheet of tissue growth promoting matrix material. Typically, the seton is thread through the elongate body or elongate sheet in a direction parallel or substantially parallel to the longitudinal axis of the elongate body or elongate sheet. Typically, the seton is thread through the elongate body or elongate sheet in a direction co-axial or substantially co-axial to the longitudinal axis of the elongate body or elongate sheet.

The elongate body or elongate sheet may be defined such that it comprises a first end and a second end, with the longitudinal axis extending from the first end to the second end. Typically, where the seton is thread through the elongate body or elongate sheet of tissue growth promoting matrix material, a portion of the seton extends beyond at least the first or the second end of the elongate body or elongate sheet. More typically, a first portion of the seton extends beyond the first end of the elongate body or elongate sheet, and a second portion of the seton extends beyond the second end.

Where the seton is thread through the elongate body or elongate sheet of tissue growth promoting matrix material, typically the seton is affixed to the elongate body or elongate sheet so as to prevent the elongate body or elongate sheet from sliding along the seton. For example, the seton may be affixed to the elongate body or elongate sheet by way of glue, knot(s), heat bonding and/or welding. In one embodiment, where the seton is thread through the elongate body or elongate sheet, the seton is affixed to the elongate body or elongate sheet via one or more knots, so as to prevent the elongate body or elongate sheet from sliding along the seton. Typically in such an embodiment, the seton is affixed to the elongate body or elongate sheet via a first knot located proximal to the first end of the elongate body or elongate sheet, the seton is thread through the elongate body or elongate sheet such that a first portion of the seton extends beyond the first end of the elongate body or elongate sheet and a second portion of the seton extends beyond the second end, and the seton is affixed to the elongate body or elongate sheet via a second knot located proximal to the second end of the elongate body or elongate sheet.

In one embodiment, the seton is affixed to the elongate sheet via a first knot located proximal to the first end of the elongate sheet, the seton is thread through the elongate sheet such that a first portion of the seton extends beyond the first end of the elongate sheet and a second portion of the seton extends beyond the second end, and the seton is affixed to the elongate sheet via a second knot located proximal to the second end of the elongate sheet, wherein the elongate sheet is rolled or folded at each of the first end and the second end, and the first and second knots are tied such that the loop of each knot encircles the respective rolled or folded portion of the elongate sheet.

In another embodiment, the elongate sheet may be formed from a first layer and a second layer of tissue growth promoting material, wherein the first layer and the second layer are affixed to each other so as to form the elongate sheet, and wherein the seton is sandwiched between the first layer and the second layer. A seton thread through an elongate sheet of tissue growth promoting material is thereby created. The first layer, the second layer and the seton may be affixed to each other, for example by way of glue, knot(s), heat bonding and/or welding.

In a further embodiment, the seton is attached to one side of the elongate body or elongate sheet of tissue growth promoting matrix material. For example, the seton may be attached to one side of the elongate body or elongate sheet by way of glue, knot(s), heat bonding and/or welding. Typically in such an embodiment, the seton is attached to one side of the elongate body or elongate sheet, such that the seton extends in a direction parallel or substantially parallel to the longitudinal axis of the elongate body or elongate sheet. Typically in such an embodiment, a portion of the seton extends beyond at least the first or the second end of the elongate body or elongate sheet. More typically, a first portion of the seton extends beyond the first end of the elongate body or elongate sheet, and a second portion of the seton extends beyond the second end.

In another embodiment, the seton is attached to one end of the elongate body or elongate sheet of tissue growth promoting matrix material. In a further embodiment, where the elongate body or elongate sheet is defined such that it comprises a first end and a second end, with a longitudinal axis extending from the first end to the second end, a first seton is attached to the first end of the elongate body or elongate sheet, and a second seton is attached to the second end of the elongate body or elongate sheet.

Where a seton is attached to one or both ends of the elongate body or elongate sheet, the seton may be attached for example by the way of glue, knot(s), heat bonding and/or welding. In one embodiment, the seton is attached to one end of the elongate body or elongate sheet of tissue growth promoting matrix material via a knot. In a further embodiment, the seton is attached to one end of the elongate sheet of tissue growth promoting matrix material via a knot, wherein the elongate sheet is rolled or folded, and the knot is tied such that the loop of the knot encircles the respective rolled or folded portion of the elongate sheet. For example, a first seton may be attached to the first end of the elongate sheet via a first knot, and a second seton may be attached to the second end of the elongate sheet via a second knot, wherein the elongate sheet is rolled or folded at each of the first end and the second end, and the first and second knots are tied such that the loop of each knot encircles the respective rolled or folded portion of the elongate sheet.

According to a second aspect of the present invention, there is provided a device suitable for treating a fistula, wherein the fistula comprises a fistula tract, wherein the device comprises a seton and an elongate body of tissue growth promoting matrix material, wherein the elongate body is suitable for being positioned within the fistula tract, and wherein the device comprises one or more attachment points positioned along the length of the elongate body, wherein the one or more attachment points are configured to connect the elongate body to a further seton.

Each attachment point may be provided in the form of a loop, ring, clip, connector or other means configured to connect the elongate body to a further seton. Each attachment point may be of the same type or different. Typically, each attachment point is of the same type.

In one embodiment of the second aspect of the invention, each attachment point is provided in the form of an attachment loop. In other words, the device comprises one or more attachment loops positioned along the length of the elongate body, wherein the one or more attachment loops are configured to connect the elongate body to a further seton. Typically, the one or more attachment loops are configured to allow the further seton to be tied to the device via the one or more loops.

As used herein, unless stated otherwise the term “loop” does not include discontinuous or broken loops.

Each attachment loop may have the same or different dimensions. Typically, each attachment loop has substantially the same dimensions. More typically, each attachment loop has the same dimensions.

In one embodiment, each attachment loop has an internal circumference from 3 to 30 mm. Typically, each attachment loop has an internal circumference from 5 to 20 mm. More typically, each attachment loop has an internal circumference from 6 to 15 mm.

In one embodiment of the second aspect of the invention, the device comprises from 2 to 50 attachment points or attachment loops positioned along the length of the elongate body. Typically, the device comprises from 5 to 30 attachment points or attachment loops positioned along the length of the elongate body. More typically, the device comprises from 10 to 20 attachment points or attachment loops positioned along the length of the elongate body.

In one embodiment of the second aspect of the invention, the elongate body comprises a first end and a second end, with a longitudinal axis extending from the first end to the second end.

Typically, one or more of the attachment points or the attachment loops are positioned on an outer surface that is parallel or substantially parallel to the longitudinal axis of the elongate body. More typically, two or more of the attachment points or the attachment loops are positioned on an outer surface that is parallel or substantially parallel to the longitudinal axis of the elongate body.

The attachment points or attachment loops may be positioned on a single outer surface or on two or more outer surfaces of the elongate body. Typically, each outer surface on which the attachment points or attachment loops are positioned is parallel or substantially parallel to the longitudinal axis of the elongate body. For example, in one embodiment the elongate body comprises a first outer surface and a second outer surface, wherein the first outer surface and the second outer surface are both parallel or substantially parallel to the longitudinal axis of the elongate body, wherein one or more attachment points or attachment loops are positioned on the first outer surface, and wherein one or more attachment points or attachment loops are positioned on the second outer surface.

In one embodiment, each outer surface on which one or more attachment points or attachment loops are positioned comprises from 1 to 25 attachment points or attachment loops. More typically, each outer surface on which one or more attachment points or attachment loops are positioned comprises from 3 to 15 attachment points or attachment loops. More typically still, each outer surface on which one or more attachment points or attachment loops are positioned comprises from 5 to 10 attachment points or attachment loops.

The elongate body of the second aspect of the invention may be defined such that it has a longitudinal axis (x) and two further axes (y) and (z) that are perpendicular to each other and perpendicular to the longitudinal axis (x). When so defined, the maximum length of the elongate body as measured along the x-axis is greater than both the maximum width and the maximum height of the elongate body as measured along the y- and z-axis respectively.

Typically, the maximum length of the elongate body as measured along the x-axis is from 2 to 30 times greater than the larger of the maximum of the width and the maximum of the height of the elongate body as measured along the y- and z-axis respectively. More typically, the maximum length of the elongate body is from 5 to 15 times greater than the larger of the maximum of the width and the maximum of the height of the elongate body. More typically, the maximum length of the elongate body is from 8 to 12 times greater than the larger of the maximum of the width and the maximum of the height of the elongate body.

In one embodiment, the maximum length of the elongate body as measured along the x-axis is from 10 to 200 mm. Typically, the maximum length of the elongate body is from 30 to 150 mm. More typically, the maximum length of the elongate body is from 50 to 100 mm.

In one embodiment, the larger of the maximum of the width or the maximum of the height of the elongate body, as measured along the y- and z-axis respectively, is from 3 to 20 mm. Typically, the larger of the maximum of the width or the maximum of the height of the elongate body is from 4 to 15 mm. More typically, the larger of the maximum of the width or the maximum of the height of the elongate body is from 5 to 10 mm.

In one embodiment, the elongate body is an elongate sheet of tissue growth promoting matrix material. In such an embodiment, the elongate sheet may be defined such that when laid flat it has a longitudinal axis (x) and two further axes (y) and (z) that are perpendicular to each other and perpendicular to the longitudinal axis (x). Typically, when laid flat the y-axis is parallel to the plane of the sheet and the z-axis is perpendicular to the plane of the sheet. When so defined, the maximum length of the elongate sheet as measured along the x-axis is greater than the maximum width of the elongate sheet as measured along the y-axis, and the maximum width of the elongate sheet as measured along the y-axis is greater than the maximum height (i.e. the thickness) of the elongate sheet as measured along the z-axis.

Typically, the maximum length of the elongate sheet as measured along the x-axis is from 2 to 30 times greater than the maximum width of the elongate sheet as measured along the y-axis. More typically, the maximum length of the elongate sheet is from 5 to 15 times greater than the maximum width of the elongate sheet. More typically, the maximum length of the elongate sheet is from 8 to 12 times greater than the maximum width of the elongate sheet.

Typically, the maximum width of the elongate sheet as measured along the y-axis is from 5 to 50 times greater than the maximum height or thickness of the elongate sheet as measured along the z-axis. More typically, the maximum width of the elongate sheet is from 10 to 30 times greater than the maximum height or thickness of the elongate sheet. More typically, the maximum width of the elongate sheet is from 15 to 20 times greater than the maximum height or thickness of the elongate sheet.

In one embodiment, the maximum length of the elongate sheet as measured along the x-axis is from 10 to 200 mm. Typically, the maximum length of the elongate sheet is from 30 to 150 mm. More typically, the maximum length of the elongate sheet is from 50 to 100 mm.

In one embodiment, the maximum width of the elongate sheet as measured along the y-axis is from 3 to 20 mm. Typically, the maximum width of the elongate sheet is from 4 to 15 mm. More typically, the maximum width of the elongate sheet is from 5 to 10 mm.

In one embodiment, the maximum height or thickness of the elongate sheet as measured along the z-axis is from 0.1 to 2 mm. Typically, the maximum height or thickness of the elongate sheet is from 0.2 to 1 mm. More typically, the maximum height or thickness of the elongate sheet is from 0.3 to 0.5 mm.

Typically, the elongate sheet of tissue growth promoting matrix material is substantially rectangular, trapezoid or oval in shape. More typically, the elongate sheet of tissue growth promoting matrix material is substantially rectangular or trapezoid in shape. Most typically, the elongate sheet of tissue growth promoting matrix material is substantially rectangular in shape. Where the elongate sheet is substantially rectangular or trapezoid in shape, the corners of the rectangle or trapezoid may optionally be rounded or chamfered.

The attachment points or attachment loops may be distributed uniformly or non-uniformly across each outer surface upon which they are positioned.

In one embodiment of the second aspect of the invention, the attachment points or attachment loops are distributed evenly or substantially evenly along the entire length of the elongate body. For example, the attachment points or attachment loops may be positioned in a first line along the length of the first outer surface of the elongate body, and a second line along the length of the second outer surface of the elongate body, wherein in each line the attachment points or attachment loops are distributed evenly or substantially evenly along the entire length of the elongate body.

Where the elongate body is an elongate sheet of tissue growth promoting matrix material, in one embodiment one or more of the attachment points or attachment loops are positioned on a surface that is parallel or substantially parallel to the x- and y-axes, i.e. any surface that is parallel or substantially parallel to the plane of the sheet when the sheet is laid flat. Typically in such an embodiment, one or more of the attachment points or attachment loops are positioned on a first surface that is parallel or substantially parallel to the x- and y-axes, and one or more of the attachment points or attachment loops are positioned on a second surface that is parallel or substantially parallel to the x- and y-axes. Thus, for example one or more of the attachment points or attachment loops may be positioned on a first side of the elongate sheet, and one or more of the attachment points or attachment loops may be positioned on a second side of the elongate sheet.

In accordance with the second aspect of the invention, the seton may be affixed to the elongate body or elongate sheet of tissue growth promoting matrix material.

In one embodiment, the seton is thread through the elongate body or elongate sheet of tissue growth promoting matrix material. Typically, the seton is thread through the elongate body or elongate sheet in a direction parallel or substantially parallel to the longitudinal axis of the elongate body or elongate sheet. Typically, the seton is thread through the elongate body or elongate sheet in a direction co-axial or substantially co-axial to the longitudinal axis of the elongate body or elongate sheet.

The elongate body or elongate sheet may be defined such that it comprises a first end and a second end, with the longitudinal axis extending from the first end to the second end. Typically, where the seton is thread through the elongate body or elongate sheet of tissue growth promoting matrix material, a portion of the seton extends beyond at least the first or the second end of the elongate body or elongate sheet. More typically, a first portion of the seton extends beyond the first end of the elongate body or elongate sheet, and a second portion of the seton extends beyond the second end.

Where the seton is thread through the elongate body or elongate sheet of tissue growth promoting matrix material, typically the seton is affixed to the elongate body or elongate sheet so as to prevent the elongate body or elongate sheet from sliding along the seton. For example, the seton may be affixed to the elongate body or elongate sheet by way of glue, knot(s), heat bonding and/or welding. In one embodiment, where the seton is thread through the elongate body or elongate sheet, the seton is affixed to the elongate body or elongate sheet via one or more knots, so as to prevent the elongate body or elongate sheet from sliding along the seton. Typically in such an embodiment, the seton is affixed to the elongate body or elongate sheet via a first knot located proximal to the first end of the elongate body or elongate sheet, the seton is thread through the elongate body or elongate sheet such that a first portion of the seton extends beyond the first end of the elongate body or elongate sheet, and a second portion of the seton extends beyond the second end, and the seton is affixed to the elongate body or elongate sheet via a second knot located proximal to the second end of the elongate body or elongate sheet.

In one embodiment, the seton is affixed to the elongate sheet via a first knot located proximal to the first end of the elongate sheet, the seton is thread through the elongate sheet such that a first portion of the seton extends beyond the first end of the elongate sheet, and a second portion of the seton extends beyond the second end, and the seton is affixed to the elongate sheet via a second knot located proximal to the second end of the or elongate sheet, wherein the elongate sheet is rolled or folded at each of the first end and the second end, and the first and second knots are tied such that the loop of each knot encircles the respective rolled or folded portion of the elongate sheet.

In another embodiment, the elongate sheet may be formed from a first layer and a second layer of tissue growth promoting material, wherein the first layer and the second layer are affixed to each other so as to form the elongate sheet, and wherein the seton is sandwiched between the first layer and the second layer. A seton thread through an elongate sheet of tissue growth promoting material is thereby created. The first layer, the second layer and the seton may be affixed to each other, for example by way of glue, knot(s), heat bonding and/or welding.

In a further embodiment, the seton is attached to one side of the elongate body or elongate sheet of tissue growth promoting matrix material. For example, the seton may be attached to one side of the elongate body or elongate sheet by way of glue, knot(s), heat bonding and/or welding. Typically in such an embodiment, the seton is attached to one side of the elongate body or elongate sheet, such that the seton extends in a direction parallel or substantially parallel to the longitudinal axis of the elongate body or elongate sheet. Typically in such an embodiment, a portion of the seton extends beyond at least the first or the second end of the elongate body or elongate sheet. More typically, a first portion of the seton extends beyond the first end of the elongate body or elongate sheet, and a second portion of the seton extends beyond the second end.

In another embodiment, the seton is attached to one end of the elongate body or elongate sheet of tissue growth promoting matrix material. In a further embodiment, where the elongate body or elongate sheet is defined such that it comprises a first end and a second end, with a longitudinal axis extending from the first end to the second end, a first seton is attached to the first end of the elongate body or elongate sheet, and a second seton is attached to the second end of the elongate body or elongate sheet.

Where a seton is attached to one or both ends of the elongate body or elongate sheet, the seton may be attached for example by the way of glue, knot(s), heat bonding and/or welding. In one embodiment, the seton is attached to one end of the elongate body or elongate sheet of tissue growth promoting matrix material via a knot. In a further embodiment, the seton is attached to one end of the elongate sheet of tissue growth promoting matrix material via a knot, wherein the elongate sheet is rolled or folded, and the knot is tied such that the loop of the knot encircles the respective rolled or folded portion of the elongate sheet. For example, a first seton may be attached to the first end of the elongate sheet via a first knot, and a second seton may be attached to the second end of the elongate sheet via a second knot, wherein the elongate sheet is rolled or folded at each of the first end and the second end, and the first and second knots are tied such that the loop of each knot encircles the respective rolled or folded portion of the elongate sheet.

In one embodiment of the second aspect of the invention, each attachment point or attachment loop is directly secured to the seton. Typically in such an embodiment, the seton is thread through the elongate body or elongate sheet of tissue growth promoting matrix material. For example, each attachment point or attachment loop may be glued, tied or welded to the seton. In one embodiment, each attachment point is provided in the form of an attachment loop, wherein each attachment loop is tied to the seton. Each attachment loop may be formed from the same or a different material to the seton. Typically, each attachment loop is formed from the same material as the seton.

In a further embodiment of the second aspect of the invention, each attachment point is provided in the form of an attachment loop, wherein each attachment loop is formed by the seton. Typically in such an embodiment, the seton is thread through the elongate body or elongate sheet of tissue growth promoting matrix material. In one embodiment, the entirety of the inner circumference of each attachment loop is defined by the seton. In another embodiment, the inner circumference of each attachment loop is defined in part by the seton and in part by the tissue growth promoting matrix material of the elongate body or sheet.

Where the seton is thread through the elongate body or elongate sheet of tissue growth promoting matrix material, the seton may be thread to follow a path such that the seton extends beyond the first end of the elongate body or elongate sheet, passes through the elongate body or elongate sheet and re-enters the elongate body or elongate sheet so as to form an attachment loop outside of the elongate body or elongate sheet, optionally passes through the elongate body or elongate sheet and re-enters the elongate body or elongate sheet one or more additional times so as to form one or more additional attachment loops, and exits so as to extend beyond the second end of the elongate body or elongate sheet.

In an exemplary embodiment of the second aspect of the invention, there is provided a device suitable for treating a fistula, wherein the fistula comprises a fistula tract, wherein the device comprises a seton and an elongate sheet of tissue growth promoting matrix material, wherein the elongate sheet is suitable for being positioned within the fistula tract, wherein the elongate sheet comprises a first end and a second end, with a longitudinal axis extending from the first end to the second end, wherein the seton extends beyond the first end of the elongate sheet, is repeatedly thread through a first side of the elongate sheet to a second side, then back though the second side of the elongate sheet to the first side, so as to form a plurality of attachment loops positioned on alternate sides along the length of the elongate sheet, wherein the seton is optionally thread back through the first side of the elongate sheet to a second side, and wherein the seton exits so as to extend beyond the second end of the elongate sheet, wherein the plurality of attachment loops are configured to tie the elongate sheet to one or more further setons.

Typically in such an exemplary embodiment, the first side of the elongate sheet forms a surface parallel or substantially parallel to the x- and y-axes when the sheet is laid flat, and the second side of the elongate sheet forms an opposing surface parallel or substantially parallel to the x- and y-axes when the sheet is laid flat.

Typically in such an exemplary embodiment, the seton is affixed to the elongate sheet via a first knot located proximal to the first end of the elongate sheet, and the seton is affixed to the elongate sheet via a second knot located proximal to the second end of the or elongate sheet. Typically, the elongate sheet is rolled or folded at each of the first end and the second end, and the first and second knots are tied such that the loop of each knot encircles the respective rolled or folded portion of the elongate sheet.

In one embodiment, the device of the first aspect of the invention is also a device according to the second aspect of the invention. Thus in one embodiment, there is provided a device suitable for treating a fistula, wherein the fistula comprises a fistula tract, wherein the device comprises a seton and an elongate body of tissue growth promoting matrix material, wherein the elongate body is suitable for being positioned within the fistula tract, wherein the elongate body comprises a plurality of protrusions positioned on at least one outer surface of said elongate body, and wherein the device comprises one or more attachment points positioned along the length of the elongate body, wherein the one or more attachment points are configured to connect the elongate body to a further seton.

In an exemplary embodiment, there is provided a device suitable for treating a fistula, wherein the fistula comprises a fistula tract, wherein the device comprises a seton and an elongate sheet of tissue growth promoting matrix material, wherein the elongate sheet is suitable for being positioned within the fistula tract, and wherein the elongate sheet comprises a first end and a second end, a first outer edge surface and a second outer edge surface, wherein a longitudinal axis extends from the first end to the second end when the elongate sheet is laid flat, wherein the first outer edge surface and the second outer edge surface are both parallel or substantially parallel to the longitudinal axis of the elongate sheet when laid flat, wherein a first plurality of protrusions is formed by serrations on the first outer edge surface, wherein a second plurality of protrusions is formed by serrations on the second outer edge surface, wherein the seton extends beyond the first end of the elongate sheet, is repeatedly thread through a first side of the elongate sheet to a second side, then back though the second side of the elongate sheet to the first side, so as to form a plurality of attachment loops positioned on alternate sides along the length of the elongate sheet, is optionally thread back through the first side of the elongate sheet to a second side, and exits so as to extend beyond the second end of the elongate sheet, and wherein the plurality of attachment loops are configured to tie the elongate sheet to one or more further setons.

Typically, the devices of the first aspect and the second aspect of the invention are configured such that the device may be formed into a loop, wherein the loop is of sufficient length to pass through the fistula tract and join up outside of the fistula tract. Preferably the loop comprises part or all of the seton and part or all of the elongate body or elongate sheet. Preferably said loop may act to secure the elongate body or elongate sheet within the fistula tract.

In one embodiment of the first or the second aspect of the invention, the elongate body or elongate sheet is flexible. Optionally, the elongate body or elongate sheet is also resilient, e.g. such that the elongate body or elongate sheet may be deformed from a substantially linear or substantially planar position into a non-linear or non-planar position respectively by the application of force, but will resume the substantially linear or substantially planar position on removal of the applied force.

The tissue growth promoting matrix material of the first or second aspect of the invention may be defined as a substance or structure which may act as a scaffold onto which and/or through which tissue may grow. Typically, tissue may grow both onto and through the scaffold.

In one embodiment of the first or the second aspect of the invention, the tissue growth promoting matrix material comprises a microscopic scaffold, i.e. the scaffold structure is not visible to the naked eye. In another embodiment of the first or second aspect of the invention, the tissue growth promoting matrix material comprises a macroscopic scaffold, i.e. the scaffold structure is visible to the naked eye. The tissue growth promoting matrix material may further comprise a mixture of macroscopic and microscopic scaffolds. For example, the macroscopic scaffold may be made from, coated with or embedded in a microscopic scaffold.

In one embodiment of the first or the second aspect of the invention, the tissue growth promoting matrix material comprises a scaffold, wherein the scaffold comprises a plurality of fibres. Said fibres may be woven or non-woven. Said fibres may be interconnected in an ordered or disordered structure. In one embodiment of the first or second aspect of the invention, the tissue growth promoting matrix material comprises a scaffold, wherein the scaffold comprises a plurality of fibres, wherein the fibres are interconnected in a non-woven disordered structure.

In an alternative embodiment, the scaffold may comprise a porous structure such as a sponge-like structure. Said porous structure may be microscopic or macroscopic.

Where a scaffold comprises a plurality of fibres, typically said fibres have an average diameter of from 0.1 to 50 μm. More typically, said fibres have an average diameter of from 1 to 10 μm. More typically still, said fibres have an average diameter of from 1.5 to 4 μm.

Where the fibres are interconnected in an ordered or disordered structure, typically the fibres are interconnected so as to form a porous structure. For example, the tissue growth promoting matrix material may comprise a scaffold, wherein the scaffold comprises a plurality of fibres, wherein the fibres are interconnected in a non-woven disordered porous structure. Where the fibres are interconnected so as to form a porous structure, typically the average pore size is from 1 to 100 μm. More typically, the average pore size is from 2 to 35 μm. More typically still, the average pore size is from 8 to 25 μm.

The average fibre diameter and the average pore size may be determined using a scanning electron microscope. Appropriate methodology is described in Hixon et al., Electrospinning, vol. 1, 2017, pp. 31-45. As used herein, unless stated otherwise the term ‘pore size’ refers to the longest internal dimension (i.e. the longest internal linear span) of a given pore, and the term ‘average pore size’ refers to the arithmetic mean of the pore sizes of at least 10 pores selected at random.

The tissue growth promoting matrix material may be constructed from biological or non-biological material, or a mixture thereof. Preferably the material promotes tissue remodelling and/or is remodelable. Preferably the material promotes angiogenesis.

In a preferred embodiment of the invention, the tissue growth promoting material is biocompatible. As used herein, the term “biocompatible materials” refers to materials which do not have unacceptable adverse effects on the subject (e.g. human or other animal) to be treated. Preferably the biocompatible materials do not have unacceptable adverse effects on the subject to be treated when left in contact with the subject for at least two weeks, more preferably for at least 4 weeks, and most preferably for at least 6 weeks.

In one embodiment of the invention, the tissue growth promoting matrix material is constructed from a biological material and/or a synthetic equivalent thereof. Typically in such an embodiment, the biological material and/or the synthetic equivalent thereof is fibrous. Typically in such an embodiment, the tissue growth promoting matrix material comprises a scaffold, wherein the scaffold comprises a plurality of fibres, wherein the fibres are constructed from biological material and/or a synthetic equivalent thereof.

The biological material may be xenogeneic, allogeneic (e.g. cadaveric), autogeneic, or a mixture thereof. Typically, where the tissue growth promoting matrix material is constructed from a biological material, the biological material is processed and/or purified, preferably so that the biological material is non-cellular.

Suitable substances which can provide a scaffold for tissue growth include fibrin; collagens such as Type I, Type II, Type III, Type IV or Type V collagen; other extracted collagenous extracellular matrix (ECM) materials such as submucosa tissue (e.g. intestinal submucosa, urinary bladder submucosa or uterine submucosa), fascial tissue, renal capsule membrane tissue, dermal tissue (e.g. dermal collagen), dura mater, pericardium tissue, serosa, peritoneum, basement membrane layers, amnion, omentum and the like.

As used herein, “fibrin” refers to a polymer formed from fibrin monomers, which themselves have been formed by the treatment of fibrinogen with thrombin.

Other suitable substances which can provide a scaffold for tissue growth include fibrous biological materials or synthetic equivalents thereof which have been cross-linked, for example using cross-linking agents such as dialdehydes, polyepoxides, dichloroalkanes and the like, to give material such as albumin crossed linked with glutaraldehyde. Cross-linking may also be achieved by the reaction of chemical groups within the fibrous biological materials or synthetic equivalents thereof, such as by dehydration, the formation of disulphide bridges and the like.

In a typical embodiment of the invention, the tissue growth promoting matrix material is constructed from a biodegradable material such as a biodegradable polymer. The biodegradable material may be a single biodegradable material, or a blend or combination of one or more biodegradable materials. Similarly, the biodegradable polymer may be a single biodegradable polymer, or a blend or combination of one or more biodegradable polymers. Typically in such an embodiment, the tissue growth promoting matrix material comprises a scaffold, wherein the scaffold comprises a plurality of fibres, wherein the fibres are constructed from a biodegradable material.

Alternatively or in addition, the tissue growth promoting matrix material may be constructed from a non-biodegradable material such as a non-biodegradable polymer. As used herein, a “biodegradable material” refers to a material that decomposes on contact with biological fluids or systems such as blood plasma, skin or sphincter muscle. Similarly a “biodegradable polymer” refers to a polymer that undergoes hydrolysis on contact with biological fluids or systems such as blood plasma, skin or sphincter muscle. A polymer that is “entirely biodegradable” refers to a polymer wherein at least one covalent bond in every link between constituent monomer units is able to undergo hydrolysis on contact with biological fluids or systems. It is preferred that after or during decomposition the material or polymer is absorbed into the body, i.e. the biodegradable material or polymer is also bioresorbable.

In contrast, a “non-biodegradable” material or polymer refers to a material or polymer that does not substantially decompose or undergo hydrolysis on contact with biological fluids or systems.

In one embodiment of any aspect of the invention, a “biodegradable” material or polymer decomposes or undergoes hydrolysis on contact with an aqueous solution of pH between 5 and 9, typically between 6 and 8, more typically about 7.

Typically, a “biodegradable” material or polymer undergoes decomposition or hydrolysis on contact with biological fluids or systems at a rate such that it takes on average at least 10 days for the material or polymer to degrade into its constituent non-biodegradable sections and/or constituent monomer units. More typically, it takes on average at least 20 days, at least 30 days, at least 40 days or at least 50 days for the material or polymer to degrade into its constituent non-biodegradable sections and/or constituent monomer units. Most typically, it takes on average at least 60 days for the material or polymer to degrade into its constituent non-biodegradable sections and/or constituent monomer units.

Typically, a “biodegradable” material or polymer undergoes decomposition or hydrolysis on contact with biological fluids or systems at a rate such that it takes on average less than 400 days for the material or polymer to degrade into its constituent non-biodegradable sections and/or constituent monomer units. More typically, it takes on average less than 200 days for the material or polymer to degrade into its constituent non-biodegradable sections and/or constituent monomer units. Most typically, it takes on average less than 100 days for the material or polymer to degrade into its constituent non-biodegradable sections and/or constituent monomer units.

Biodegradable polymers suitable for use in the present invention include but are not limited to polyesters such as poly-lactic acids (polylactides), polyglycolic acids (polyglycolides), polycaprolactones, polycaprolactone diols, and polycaprolactone triols; polyanhydrides such as poly(sebacic acids), poly(adipic acids), poly(fumaric anhydrides), poly(stilbene dicarboxylic acid anhydrides) and poly[1,6-bis(p-carboxy-phenoxy)hexane]; polyphosphoesters such as poly[1,4-bis(hydroxyethyl)-terephthalate-alt-ethyloxyphosphate]; polyphosphazenes such as poly(bis(1,4-dioxapentyl)phosphazenes), poly(bis(4-carboxyphenoxy)phosphazenes) and poly-[bis(1-(ethoxycarbonyl)-2-phenylethylamino)phosphazene]; polyethers such as polypropylene oxides and polyethylene glycols; other synthetic polymers such as polycarbonates, polycyanoacrylates, polydioxanones, poly(1,5-dioxepan-2-one), polyaminoacids, polyamides, polyhydroxybutyrates, polyhydroxyvalerates, polyesteramides, polyvinyl pyrrolidone, polyurethanes, polyalkylene succinates, poly(malic acid), polyalkylene oxalates, polyorthocarbonates, polyorthoesters, polyamines, polyhydroxycelluloses, polyvinyl alcohol, polyacetals, polyketals and cyclodextrins; natural polymers such as albumin, chitin, chitosan, collagen, dextran, fibrin, fibrinogen, gelatine, polysaccharides, carrageenan, tragacanth, acacia, xanthan gum and poly(alginic acid); and any combination thereof.

Biodegradable polymers can also include copolymers of any of the above, including alternating copolymers, periodic copolymers, random copolymers and block copolymers. Examples of such copolymers include poly(lactic acid-co-glycolic acids), poly(lactide-co-glycolides), poly(lactide-co-caprolactones), poly(lactide-co-caprolactone-co-glycolide), poly[(lactide-co-ethylene glycol)-co-ethyloxyphosphate], poly[(1,6-bis(p-carboxyphenoxy)hexane)-co-sebacic acid], poly(hydroxybutyric acid-co-hydroxyvaleric acid), poly[1,4-bis(hydroxyethyl)terephthalate-alt-ethyloxy-phosphate]-co-1,4-bis(hydroxyethyl)terephthalate-co-terephthalate, poly(ethylene glycol)-poly(caprolactone) methyl ether block copolymers, poly(ethylene glycol)-polylactide methyl ether block copolymers, poly(ethylene glycol)methyl ether-poly-lactide polylactide block copolymers, poly(ethylene oxide)-polycaprolactone block copolymers, poly(ethylene oxide)-polylactide block copolymers, polycaprolactone-polytetrahydrofuran-polycaprolactone block copolymers, polylactide-poly(ethylene glycol)-polylactide block copolymers, polyoxyethylene-polypropylene block copolymers, and any combination thereof.

Suitable non-biodegradable polymers for use in the present invention include celluloses such as cellulose ethers, ethyl celluloses, hydroxypropyl methyl celluloses, hydroxypropyl celluloses, hydroxyethyl celluloses, hydroxyethylmethyl celluloses, methyl celluloses, cellulose acetates and their derivatives and copolymers thereof.

Other suitable non-biodegradable polymers include polyalkylenes, polyacrylates, polymethacrylates, polypyrrolidones, polyoxyethylenes, polyoxyethylene-polypropylene copolymers, polymethylmethacrylatyes, polybutylmethacrylates, polysiloxanes, shellac, acrylic and methacrylic acid based polymers, and copolymers thereof.

In an exemplary embodiment of the first and second aspects of the invention, the tissue growth promoting matrix material comprises a scaffold, wherein the scaffold comprises a plurality of fibres, wherein the fibres are constructed from a biodegradable polymer. Typically in such an embodiment, the fibres are constructed from a biodegradable and bioresorbable polymer. For example, the fibres may be constructed from a biodegradable polyester, optionally selected from the group consisting of poly-lactic acids (polylactides), polyglycolic acid (polyglycolides), polycaprolactones, polycaprolactone diols, polycaprolactone triols, and co-polymers thereof. More typically in such an embodiment, the fibres are constructed from a biodegradable polyester selected from the group consisting of poly-lactic acids (polylactides), polyglycolic acids (polyglycolides), and co-polymers thereof. Most typically, the fibres are constructed from a poly-lactic acid such as poly-L-lactic acid (PLLA).

In one aspect of the above embodiment, the tissue growth promoting matrix material comprises a scaffold, wherein the scaffold comprises a plurality of fibres, wherein the fibres are interconnected in a non-woven disordered porous structure, and wherein the fibres are constructed from a biodegradable polymer, such as any listed in the paragraph above. Typically in such an aspect of the embodiment, the tissue growth promoting matrix material is prepared by electrospinning a solution of the polymer so as to form the scaffold. Such electrospinning procedures are described for instance in patent application WO 2007/132186, the contents of which are incorporated herein by reference in their entirety.

Optionally the tissue growth promoting matrix material of the first or second aspect of the invention may be coated or impregnated with one or more tissue growth promoting agents, and/or one or more other pharmaceutical agents. Exemplary tissue growth promoting agents include growth factors such as basic fibroblast growth factor (FGF-2), transforming growth factor beta (TGF-beta), epidermal growth factor (EGF), cartilage derived growth factor (CDGF), platelet derived growth factor (PDGF), insulin-like growth factors I and II (IGF-I and IGF-II), interferons (e.g. interferon α, β, γ) and the like. Other pharmaceutical agents may be selected from the group consisting of from anti-inflammatoires, anti-bacterial agents, immunomodulators or combinations thereof. Examples of suitable agents are listed on pages 13 to 19 of patent application WO 2011/151659, the contents of which are incorporated herein by reference in their entirety.

In any embodiment of any aspect of the invention, it is preferred that the seton and/or the device as a whole is made from biocompatible materials. For instance, the seton may be formed from flexible materials such as rubber, silicone, silk, flexible plastics such as polypropylene and the like. Typically, the seton is made from flexible plastic.

In one embodiment of the first or the second aspect of the invention, the seton is formed from one or more biodegradable materials such as biodegradable polymers.

Examples of suitable biodegradable polymers are listed above. Typically, the seton is formed from one or more biodegradable and bioresorbable polymers. Typically, the seton is formed from one or more biodegradable polyesters, optionally selected from the group consisting of poly-lactic acids (polylactides), polyglycolic acid (polyglycolides), polycaprolactones, polycaprolactone diols, polycaprolactone triols, and co-polymers thereof.

In one embodiment of the first or the second aspect of the invention, the seton comprises a braided cord coated with at least one coating. Typically in such an embodiment, the seton comprises a braided cord constructed from a first biodegradable polymer, and at least one coating, wherein the coating comprises a second biodegradable polymer and optionally a fatty acid, a fatty acid ester, or a salt thereof.

Typically, the first biodegradable polymer and the second biodegradable polymer are both biodegradable polyesters, optionally selected from the group consisting of poly-lactic acids (polylactides), polyglycolic acid (polyglycolides), polycaprolactones, polycaprolactone diols, polycaprolactone triols, and co-polymers thereof. The first biodegradable polymer and the second biodegradable polymer may be the same or different. Typically, the first biodegradable polymer and the second biodegradable polymer are different. In one example, the first biodegradable polymer is selected from the group consisting of poly-lactic acids (polylactides), polyglycolic acid (polyglycolides), and co-polymers thereof, and the second biodegradable polymer is selected from the group consisting of polyglycolic acid (polyglycolides), polycaprolactones, polycaprolactone diols, polycaprolactone triols, and co-polymers thereof. Typically, the first biodegradable polymer is a copolymer of glycolide and lactide, more preferably of glycolide and L-lactide. Typically, such a copolymer comprises about 80-95 mol% glycolide and about 5-20 mol% L-lactide. Most preferably, the copolymer comprises approximately 90 mol% glycolide and approximately 10 mol% L-lactide. Typically, the second biodegradable polymer is a copolymer of glycolide and caprolactone. Typically, the fatty acid, fatty acid ester, or salt thereof is a lactylic ester of a fatty acid, or a salt thereof. More typically, the fatty acid, fatty acid ester, or salt thereof is a stearoyl-lactylate such as stearoyl-2-lactylate or a salt thereof. Most typically, the fatty acid, fatty acid ester, or salt thereof is calcium stearoyl-2-lactylate. Coated braided cords comprising a copolymer of glycolide and lactide as the first biodegradable polymer, a copolymer of glycolide and caprolactone as the second biodegradable polymer and calcium stearoyl-2-lactylate as the fatty acid, fatty acid ester, or salt thereof are available commercially under the trade name Polysorb™ and may be used as the seton in accordance with the first or second aspect of the invention.

Typically, any parts of the seton and/or the elongate body or elongate sheet to be positioned within the fistula do not contain exposed metal. More typically, any parts of the seton and/or the elongate body or elongate sheet to be positioned within the fistula do not contain metal. Most typically, the device of the first or the second aspect of the invention does not contain metal.

The seton is elongate in shape. Typically, in any embodiment of any aspect of the invention, the overall length of the seton(s) and the elongate body or elongate sheet (e.g. when the seton is thread through the elongate body or elongate sheet) is from 100 to 1000 mm. More typically, the overall length of the seton(s) and the elongate body or elongate sheet is from 200 to 800 mm. More typically still, the overall length of the seton(s) and the elongate body or elongate sheet is from 400 to 600 mm.

In one embodiment, the seton is approximately polygonal (e.g. approximately rectangular, pentagonal, hexagonal, heptagonal or octagonal) in cross-section. In another embodiment, the seton is approximately circular in cross-section.

Typically, the cross-section of the seton has a maximum width or diameter of between 0.05 and 6 mm. More typically, said cross-section has a maximum width or diameter of between 0.1 and 4 mm. Typically, the cross-section of the seton has a minimum width or diameter of between 0.2 and 2 mm. More typically, said cross-section has a minimum width or diameter of between 0.3 and 0.5 mm.

A third aspect of the invention provides a method of manufacturing a device according to the first and/or the second aspect of the invention, said method comprising the step of affixing the seton to the elongate body or elongate sheet.

In one embodiment of the third aspect of the invention, the method is a method of manufacturing a device according to the first aspect of the invention. Typically in such an embodiment, the method comprises the steps of forming the plurality of protrusions on at least one outer surface of the elongate body and affixing the elongate body to the seton. More typically, the method comprises the steps of cutting an elongate sheet of tissue growth promoting matrix material from a larger sheet of tissue growth promoting matrix material, such that the elongate sheet is formed with one or more castellated or serrated outer edges, and affixing the elongate sheet to the seton.

In one embodiment of the third aspect of the invention, the method is a method of manufacturing a device according to the second aspect of the invention. Typically in such an embodiment, the step of affixing the seton to the elongate body or elongate sheet comprises threading the seton in and out of the elongate body or elongate sheet so as to form one or more of the attachment loops.

In an exemplary embodiment of the third aspect of the invention, the method comprises the steps of:
(i) cutting an elongate sheet of tissue growth promoting matrix material from a larger sheet of tissue growth promoting matrix material, optionally such that the elongate sheet is formed with one or more castellated or serrated outer edges that extend in a direction parallel or substantially parallel to the longitudinal axis of the elongate sheet;
(ii) folding the elongate sheet in alternate directions so as to create an accordion fold, wherein the creases of the accordion fold extend in a direction perpendicular or substantially perpendicular to the longitudinal axis of the elongate sheet;
(iii) passing the seton through the accordion folded elongate sheet, optionally with the assistance of a needle, such that the seton passes though the elongate sheet multiple times;
(iv) extending the accordion folded elongate sheet along a length of the seton; and
(v) optionally tying or otherwise securing the seton to the elongate sheet so as to prevent the elongate sheet from sliding along the seton.

Typically in steps (ii) and (iii), sharp folds or sharp creases in the elongate sheet are avoided.

A fourth aspect of the invention provides a device according to the first and/or the second aspect of the invention, for use in medicine. Typically, said device is for use in the treatment of a fistula such as an anal fistula or a recto-vaginal fistula. Typically, the treatment comprises inserting the device into the fistula tract. Typically, the treatment comprises forming the device into a loop wherein the loop passes through the fistula tract and joins up outside of the fistula tract. More typically, said loop acts to secure the elongate body or the elongate sheet within the fistula tract.

According to a fifth aspect of the present invention, there is provided a method of treating a fistula comprising the use of a device according to the first and/or the second aspect of the invention. Typically, the fistula is an anal fistula or a recto-vaginal fistula. Typically, the method comprises inserting the device into the fistula tract. Typically, the method comprises forming the device into a loop wherein the loop passes through the fistula tract and joins up outside of the fistula tract. More typically, said loop acts to secure the elongate body or the elongate sheet within the fistula tract.

As used herein, a “fistula” refers to any abnormal passage or communication through the body between two epithelial surfaces, including those occurring naturally, e.g. as a result of infection, those occurring as a result of injury, e.g. as a result of impalement, and those man-made, for example as a result of surgery or body piercing.

In one embodiment of any aspect of the present invention, the fistula to be treated is selected from:
(i) a body piercing or skin-to-skin fistula;
(ii) an anal or anorectal fistula, which may be classified anatomically as intersphincteric, transphincteric, suprasphincteric or extrasphincteric;
(iii) a recto-vaginal fistula such as an anovulval, anovaginal, rectovulval, rectovaginal or rectovestibular fistula, wherein the recto-vaginal fistula may be classified anatomically as infrasphincteric, transphincteric, or suprasphincteric;
(iv) a recto-prostatic fistula;
(v) a gastrointestinal fistula such as a tracheo-oesophageal, gastro-cutaneous, ileo-cutaneous, colo-cutaneous, recto-cutaneous, colo-vaginal or gastrointestinal-vascular fistula;
(vi) a urinary fistula such as a urethrocutaneous, urethrovaginal, urethrovesical, vesciovaginal, rectovesical or rectourethral fistula; or
(vii) a fistula comprising a combination of any of the aforementioned fistulas, such as a recto-vesico-vaginal fistula.

Typically said fistulas are complete (i.e. both ends open on a mucosal or exterior surface of the body). Complete fistulas may be external (i.e. between a hollow organ and an external surface of the body) or bimucosal (i.e. both ends open on a mucosal surface of the body).

In one embodiment, said fistulas are simple (i.e. contain no blind tracts and contain only one opening at each end of the tract). Optionally however said fistulas include blind tracts and/or are complex (i.e. include more than two openings due to division of the tract). An example of a complex fistula is a horseshoe fistula (where two ends of the fistula tract open on an exterior surface of the body and a third end opens into a hollow organ such as the anal canal).

In one embodiment of the fourth or fifth aspect of the invention, the fistula to be treated is complex and the device is in accordance with at least the second aspect of the invention. Typically in such an embodiment, the treatment or method comprises the steps of:
(i) securing a further seton to an attachment point or attachment loop of the device of the second aspect of the invention, optionally wherein the further seton forms part of an additional device according to the first and/or second aspect of the invention;
(ii) inserting the combined device and further seton (or additional device) into the branched tract of the complex fistula, such that one or more elongate bodies or elongate sheets are positioned within the branched fistula tract, and such that a first end of a seton of the device emerges from a first opening at a first end of the branched fistula tract, a second end of a seton of the device emerges from a second opening at a second end of the branched fistula tract, and one end of the further seton emerges from a third opening at a third end of the branched fistula tract; and
(iii) securing two or more of the emerged ends of the seton(s) to each other, e.g. by tying the emerged ends together in one or more knots.

Typically, the fistula to be treated is selected from an anal fistula or a recto-vaginal fistula. More typically, the fistula is an anal fistula.

Typically, the patient to be treated in any of the preceding aspects of the present invention is a human. Typically, the patient to be treated is in need of such treatment. Optionally, the patient may also be suffering from an inflammatory bowel disease such as Crohn's disease.

For the avoidance of doubt, insofar as is practicable any embodiment of a given aspect of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

### DETAILED DESCRIPTION OF THE INVENTION

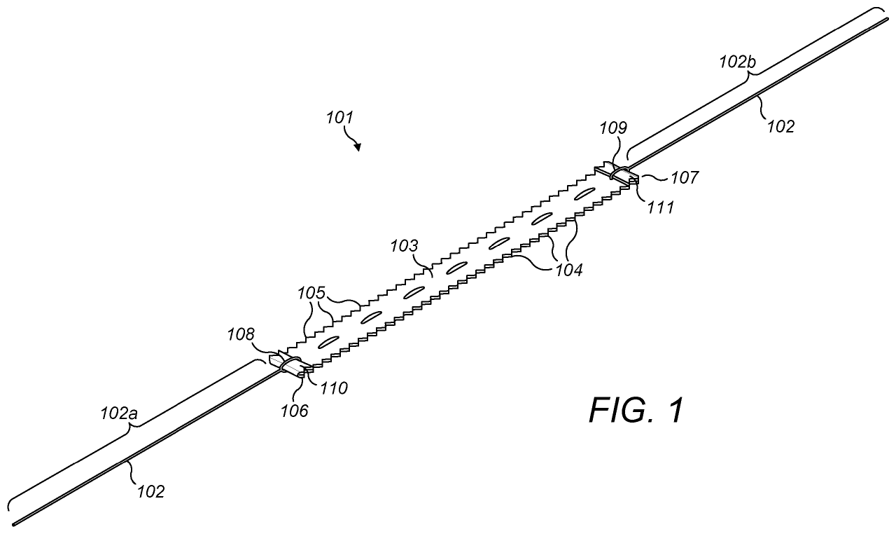

Embodiments of the present invention will now be described by way of example only, with reference to the accompanying drawings, in which:
FIG. 1 shows a perspective view of a device according to the invention comprising a seton and an elongate sheet of tissue growth promoting matrix material.

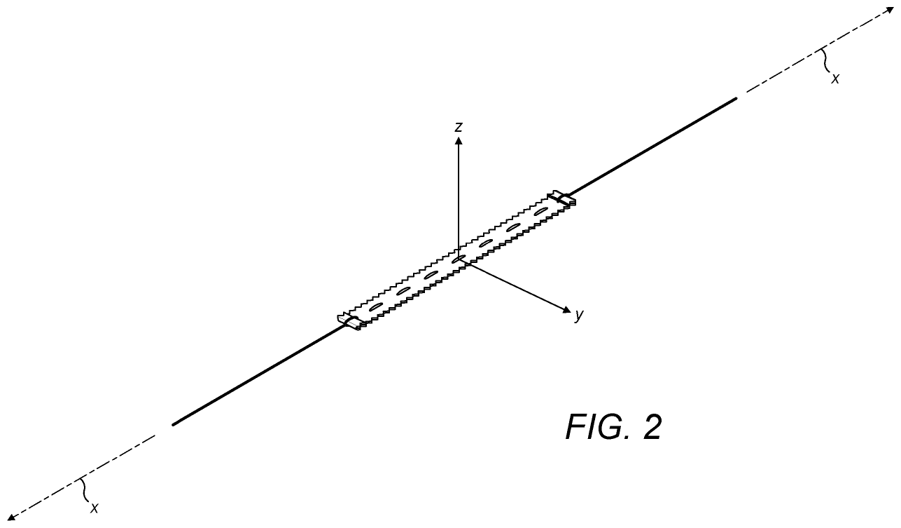

FIG. 2 shows a perspective view of the device according to FIG. 1 illustrating the longitudinal axis and various dimensions of the device.

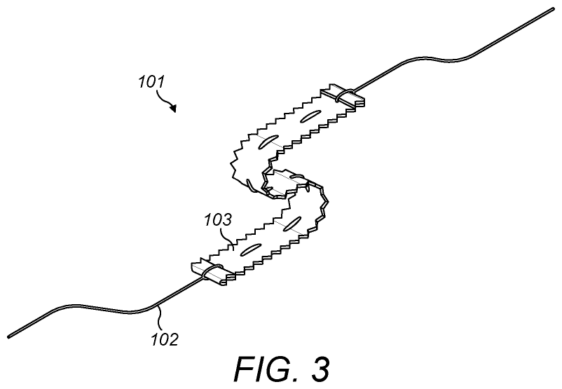

FIG. 3 shows a perspective view of the device according to FIG. 1 in a non-linear position.

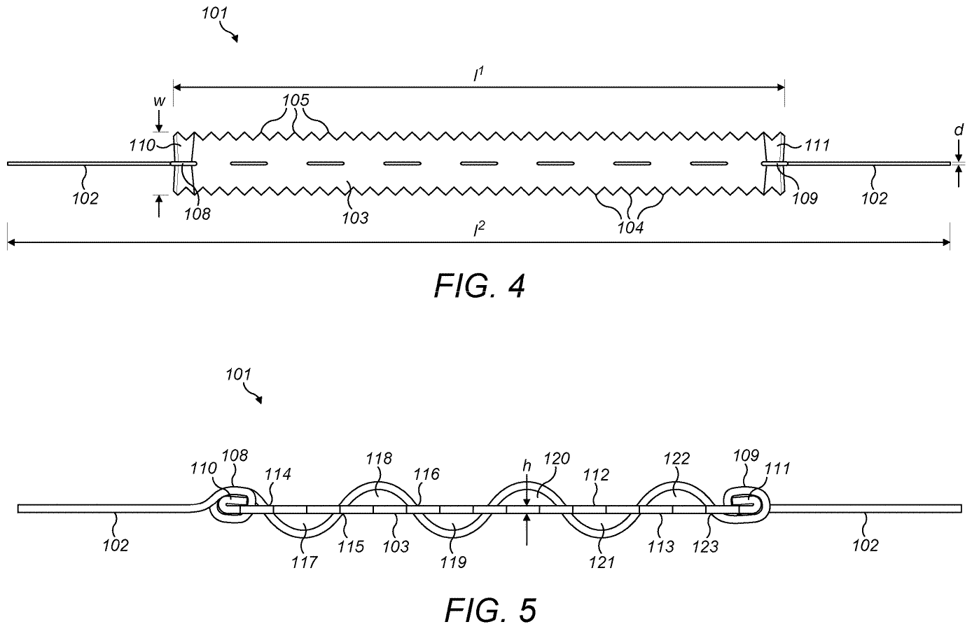

FIG. 4 shows a top view of the device according to FIG. 1

FIG. 5 shows a side view of the device according to FIG. 1

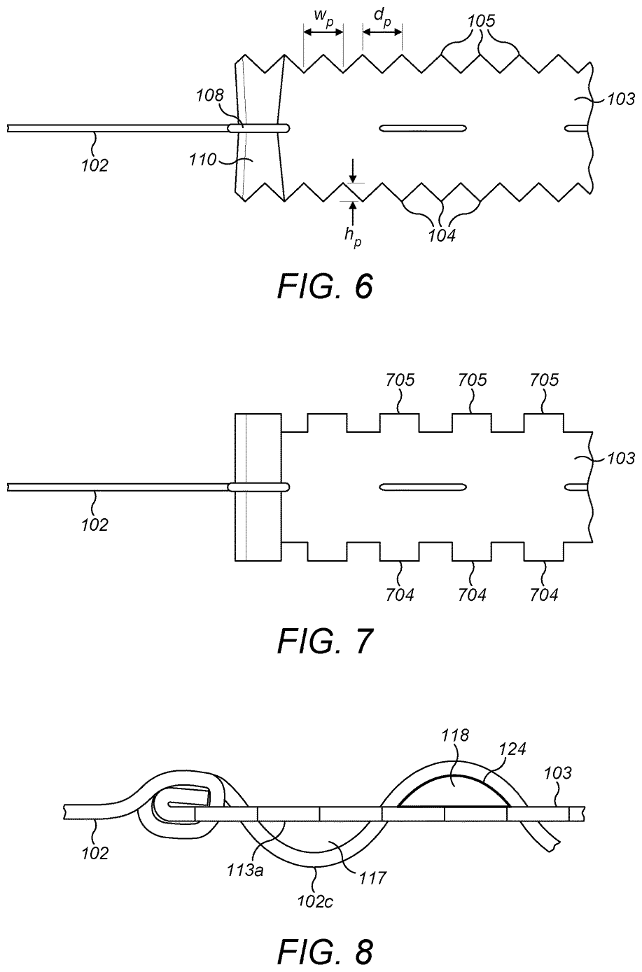

FIG. 6 shows an expanded top view of a terminal portion of the elongate sheet of the device of FIG. 1.

FIG. 7 shows an expanded top view of a terminal portion of the elongate sheet of the device of FIG. 1 but with castellated protrusions.

FIG. 8 shows an expanded side view of a terminal portion of the elongate sheet of the device of FIG. 1.

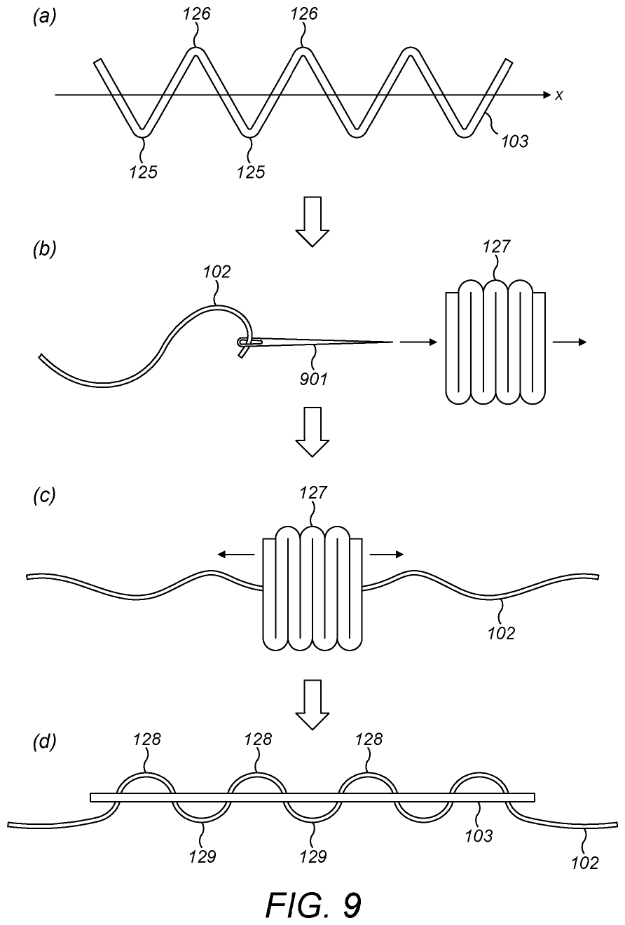

FIG. 9 shows a side view illustrating a manufacturing method of the device of FIG. 1.

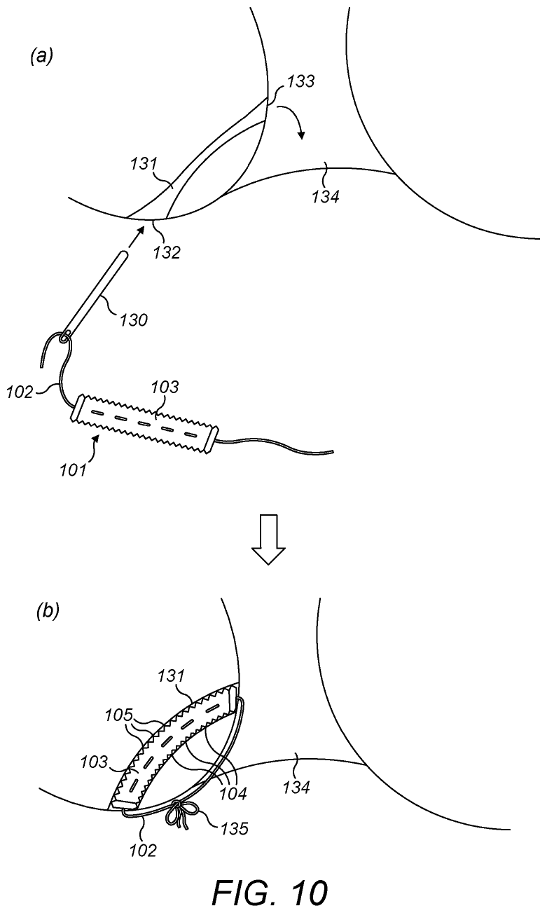

FIG. 10 shows a schematic diagram of the device of FIG. 1 being positioned within a simple anal fistula.

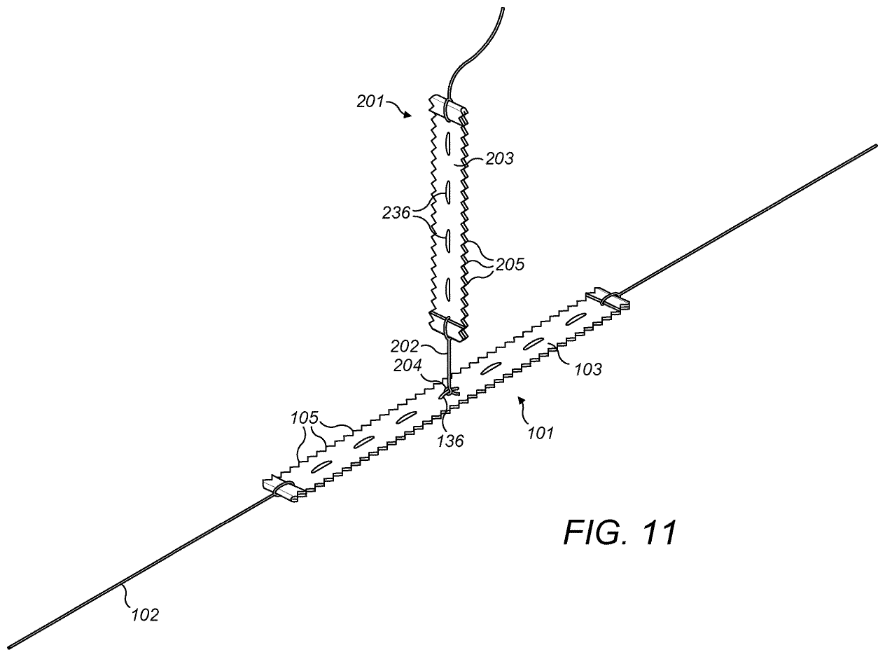

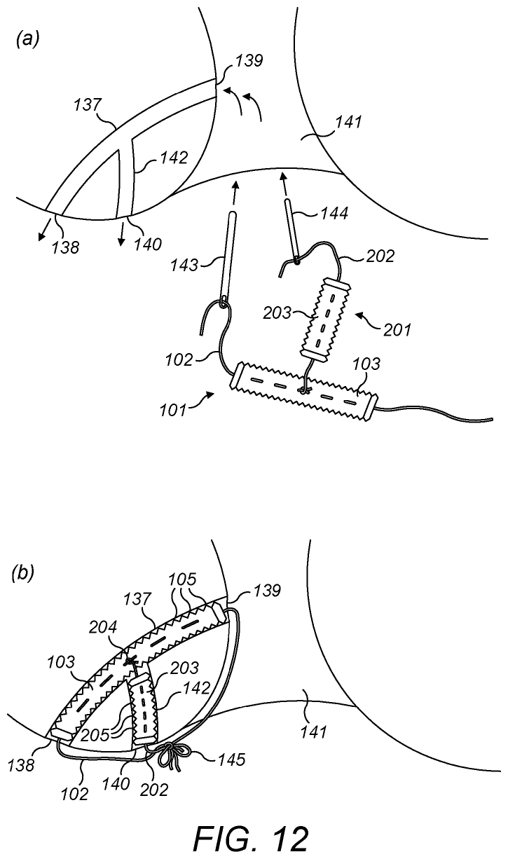

FIG. 11 shows a perspective view of a device of the invention tied to a further device of the invention via an attachment loop.

FIG. 12 shows a schematic diagram of the tied devices of FIG. 11 being positioned within a complex anal fistula.

### KEY TO FIGURES

101 Device
102 Seton
102a First portion of the seton
102b Second portion of the seton
102C Seton portion
103 Elongate sheet of tissue growth promoting matrix material
104 First plurality of protrusions
105 Second plurality of protrusions
106 First end of the elongate sheet
107 Second end of the elongate sheet
108 First knot
109 Second knot
110 First double thickness region
111 Second double thickness region
112 First side of the elongate sheet
113 Second side of the elongate sheet
113a Portion of the side of the elongate sheet
114 Position on elongate sheet
115 Position on elongate sheet
116 Position on elongate sheet
117 Attachment loop
118 Attachment loop
119 Attachment loop
120 Attachment loop
121 Attachment loop
122 Attachment loop
123 Position on elongate sheet
124 Inner circumference of attachment loop
125 Crease
126 Crease
127 Compressed elongate sheet
128 Attachment loop
129 Attachment loop
130 Probe
131 Fistula tract
132 First opening
133 Second opening
134 Anal canal
135 Knot
136 Attachment loop of first device
137 Complex fistula tract
138 First opening
139 Second opening
140 Third opening
141 Anal canal
142 Branch of fistula tract
143 First probe
144 Second probe
145 Knot
201 Further device
202 Seton of further device
203 Elongate sheet of tissue growth promoting matrix material of further device
204 Knot
205 Plurality of protrusions of further device
236 Attachment loop of further device
704 Castellated protrusions
705 Castellated protrusions
901 Needle

The devices of the present invention are particularly useful for the treatment of fistulas such as anal or recto-vaginal fistulas. Depending on the fistula to be treated, it may be desirable and/or necessary to pre-treat the fistula prior to the use of the devices of the present invention that comprise an elongate body of tissue growth promoting matrix material. For instance, if the fistula is heavily infected, it may be desirable to insert a drainage seton according to the prior art methods discussed above. The drainage seton may optionally incorporate antibiotics and/or anti-inflammatoirer to help reduce the extent of infection. Alternatively, it may be preferable to insert instead a device comprising a fistula stent, such as a device described in WO 2014/023962.

After a period of time, the drainage seton or the fistula stent may be removed prior to the insertion of a device comprising an elongate body of tissue growth promoting matrix material according to the present invention.

Optionally, prior to the insertion of a device according to the present invention, the fistula tract is cleaned, for example using a jet of water and/or a suitable brush.

Referring now to FIGS. 1, 4 and 5, there is shown a device 101 according to an embodiment of the present invention, wherein the device comprises a seton 102 and an elongate body of tissue growth promoting matrix material 103. The elongate body can be seen as rectangular prismatic in form and is provided by a sheet of tissue growth promoting matrix material. Hence, the elongate body may be seen as an elongate sheet of tissue growth promoting matrix material 103.

As illustrated in FIG. 2, when laid flat and outstretched, the device and the elongate sheet may be seen to have a longitudinal axis x that extends lengthwise though the seton 102 and the elongate sheet of tissue growth promoting matrix material 103. As illustrated, the device may further be seen to have a y-axis that is perpendicular to the longitudinal x-axis and parallel to the plane of the sheet, and a z-axis that perpendicular to both the x-axis and the y-axis.

As will be appreciated, as used herein all reference to the longitudinal axis and associated orientations and dimensions refer to the device when laid out flat in an outstretched position, as illustrated in FIGS. 1 and 2. Unless stated otherwise, the dimensions are measured in such a position. It will be understood however, that both the seton 102 and the elongate sheet of tissue growth promoting material 103 are flexible and may adopt a wide variety of positions, such as that shown in FIG. 3.

As discussed in the summary of the invention, the dimensions of the elongate body or sheet 103 and the seton 102 may vary depending on the fistula that is to be treated.

Typically however, the maximum length 11 of the elongate sheet 103 as measured along the x-axis (see FIG. 4) is about 75 mm, the maximum width w of the elongate sheet as measured along the y-axis (see FIG. 4) is about 7.5 mm, and the maximum height or thickness h as measured along the z-axis (see FIG. 5) is about 0.4 mm. Similarly, the seton 102 typically has a maximum width or diameter d of about 0.35 mm (see FIG. 4), and the seton is selected such that the overall length 12 of the device 101 is about 475 mm (see FIG. 4). In the embodiment shown, the seton is approximately circular in cross-section, although it will be appreciated that other cross-sectional shapes are possible.

In the embodiment described, the tissue growth promoting matrix material of the elongate sheet 103 comprises a scaffold, wherein the scaffold comprises a plurality of fibres, wherein the fibres are interconnected in a non-woven disordered structure. The fibres are formed from poly-L-lactic acid (PLLA) which is a biodegradable, biocompatible and bioresorbable polymer. The matrix material was purchased from Neotherix Limited and is formed by electrospinning a solution of the polymer in 1,1,1,3,3,3-hexafluoropropan-2-ol (HFIP), so as to generate material having a fibre diameter of 2-3 μm and an average pore size of about 10 μm. While as discussed above the use of other tissue growth promoting matrix materials is possible, the use of an electrospun biodegradable polymer such as that exemplified allows for excellent tissue growth promoting properties due to the large surface area and porosity facilitating both tissue ingrowth and drainage while the polymer fibres degrade over time and are absorbed and ultimately excreted by the body.

In the embodiment described, the seton 102 comprises a braided cord having an outer coating. The braided cord is formed from a poly(lactide-co-glycolide) (PLGA) copolymer having a lactide content of approximately 10 mol % and a glycolide content of approximately 90 mol %. The outer coating is formed from a poly(caprolactone-co-glycolide) copolymer in admixture with calcium stearoyl-2-lactylate. Again, the copolymers are biodegradable, biocompatible and bioresorbable. Such setons are commercially available as sutures under the trade name PolysorbTM. While the use of setons made from other materials is envisaged, such coated braided cords offer the advantages of strength and flexibility, while at the same time being sufficiently smooth to facilitate their use and being biodegradable so as to avoid the need for subsequent surgical removal.

Turning back to FIG. 1, it can be seen that the elongate body or sheet 103 comprises a first plurality of protrusions 104 positioned on a first outer edge surface that is substantially parallel to the longitudinal axis x of the elongate sheet, and a second plurality of protrusions 105 positioned on a second outer edge surface that is also substantially parallel to the longitudinal axis x of the elongate sheet. Typically, about 40 protrusions are provided in the first plurality of protrusions 104, and about 40 protrusions are provided in the second plurality of protrusions 105. The protrusions are of unitary construction with the elongate sheet 103, and are provided in the form of serrations formed by cutting the outer edge of the elongate sheet accordingly. As illustrated in FIG. 6, the distance dp between the maxima of each neighbouring protrusion is typically about 2 mm, the width or maximum distance (Wp) that each protrusion extends between two neighbouring minima is typically about 2 mm, and the height or maximum distance (hp) that each protrusion extends from a neighbouring minima of the surface is typically about 1 mm.

When the device is inserted into the fistula tract, the protrusions 104, 105 may act to grip or dig into the inner wall of the fistula. Accordingly, the protrusions may serve to prevent both rotational movement of the elongate body or sheet 103 within the fistula tract, and prevent lateral or sideways movement so as to prevent the elongate body or sheet 103 from sliding out of the fistula tract. Since the protrusions help maintain the elongate body or sheet 103 in a single position relative to the surrounding tissue of the fistula tract, tissue ingrowth is encouraged.

As will be appreciated, the protrusions need not be triangular or serrated in shape, and could for example be castellated as illustrated by protrusions 704 and 705 in FIG. 7. Other shapes are described in the summary of the invention above. Advantageously however, each protrusion comprises at least one point, which may serve to better grip or dig into the inner wall of the fistula.

As illustrated in FIGS. 6 and 7, the minima between neighbouring protrusions 105, 705, is in the form of a vee-shape (FIG. 6) or squared profile with sharp or pointed internal corners (FIG. 7). It may be desirable, however, to ensure that each minima between neighbouring protrusions is not pointed or vee-shaped. For example, each minima between neighbouring protrusions may be curved or rounded, e.g. so as to form a concave curve between the maxima of each neighbouring protrusion. Tear promotion points are thereby minimised.

As best illustrated in FIGS. 1, 4 and 5, the seton 102 is thread through the elongate sheet 103, such that a first portion 102a of the seton 102 extends beyond a first end 106 of the elongate sheet 103, a second portion 102b of the seton 102 extends beyond a second end 107, and the seton 102 spans the entire length of the elongate sheet 103.

Since in use the elongate sheet 103 is placed in the fistula tract and the first and second portions 102a and 102b are tied together outside of the fistula tract so as to form a loop or seton stitch, such a configuration means that the securement relies entirely on the robust seton cord for strength and is not reliant on potentially weaker materials such as the tissue growth promoting matrix material. More robust securement is thereby achieved.

The elongate sheet 103 is folded back on itself at the first end so as to create a double thickness region of the sheet 110. Using the seton, a first knot 108 is tied about the folded portion 110 of the elongate sheet. Similarly, the elongate sheet 103 is folded back on itself at the second end so as to create another double thickness region of the sheet 111. Using the seton, a second knot 109 is tied about the folded portion 111 of the elongate sheet. As will be appreciated, the two knots prevent the elongate sheet 103 from sliding along the seton 102. Moreover, by folding or rolling the elongate sheet, a more robust connection is formed, enabling the surgeon or care practitioner to pull forcibly on either of the first or second portion of the seton (102a or 102b) with minimal risk of the elongate sheet 103 tearing. The same folds or rolls also minimise the risk of the elongate sheet 103 tearing when in situ due to the everyday strains and stresses imparted by the patient's movement.

In the embodiments illustrated, the elongate sheet 103 of tissue growth promoting matrix material is substantially rectangular in shape (see especially the top view of the device 101 in FIG. 4), and the corners of the rectangle at the first end 106 and the second end 107 each form a right angle. It may be desirable however, to ensure that the corners of the rectangle are rounded or chamfered, thus aiding insertion of the device into the fistula tract.

Turning to FIG. 5 and following the path of the seton 102 from the first knot 108, it can be seen that the seton is thread through a first side 112 of the elongate sheet 103 at position 114 to a second side 113, then back through the second side 113 at position 115, then back though the first side 112 at position 116, and so on down the length of the elongate sheet. In so doing, it can be seen that a plurality of attachment loops 117, 118, 119, 120, 121, 122 are formed, positioned on alternate sides along the length of the elongate sheet, in a direction substantially parallel to the longitudinal x-axis. As shown, it can be seen that before the second knot 109, the seton 102 passes through the elongate sheet 103 at position 123 for the last time from the first side 112 to the second side 113. However, this is not critical and the last pass though the elongate sheet may equally be from the second side to the first side. It will be appreciated that as illustrated in FIGS. 5, 8 and 9, the seton 102 is shown loosely thread so as to best illustrate the path through the elongate sheet 103 and the attachment loops 117, 118, etc. In practice however, the seton 102 and elongate sheet 103 will typically be pulled taught before completing the tying of the first and second knots 108, 109, such that the seton lies flat against the surfaces 112, 113 of the elongate sheet 103.

In the embodiment shown, the distances between the positions 114, 115, 116, etc. at which the seton passes though the elongate sheet are approximately equally spaced. Consequently, the attachment loops 117, 118, 119, 120, 121, 122, etc. are distributed substantially evenly along the entire length of the elongate sheet and are each of substantially the same size. It will be appreciated however that alternate spacings may be used, so as to vary the distribution and size of the attachment loops. Similarly, in the embodiment shown in FIG. 1 there are seven attachment loops on the first side of the elongate sheet and eight attachment loops on the second side, thus resulting in 15 attachment loops in total positioned along the length of the elongate sheet. Again however, it will be appreciated that the number of attachment loops on each side and in total may vary.

The plurality of attachment loops 117, 118, 119, 120, 121, 122 are configured to tie the elongate sheet to one or more further setons. As best shown in FIG. 8, the inner circumference of the attachment loop 117 is formed in part by the seton portion 102c and in part by the portion 113a of the side of the elongate sheet 103. Thus, in relation to the attachment loop 118, the inner circumference is illustrated by the bold line 124. The inner circumference is advantageously chosen such that a further seton can be passed though the attachment loop by the end user and tied relatively easily, while at the same time substantial lateral movement of the tied further seton is prevented. To achieve this, an inner circumference of 10 mm is typical.

Turning now to the manufacture of the device of FIG. 1, it will be understood that this typically takes place in a factory, prior to delivery to the surgeon or other end user. The elongated sheet 103 may be cut from a larger sheet of the tissue growth promoting matrix material. Typically, the serrations or castellations are cut at the same time as the elongate sheet is cut from the larger sheet, thus permitting the cutting of multiple elongate sheets with minimal wastage, especially if the same cut is used to form the serrations or castellations on more than one elongate sheet. The cutting method is not limited any may include, for example die cutting, laser cutting, or the use of scissors.

In a particularly efficient method of manufacture, illustrated in FIG. 9, the elongate sheet 103 may be folded in in alternate directions so as to create an accordion fold, wherein the creases 125, 126 of the accordion fold extend in a direction perpendicular or substantially perpendicular to the longitudinal x-axis of the elongate sheet. The accordion folded sheet may then be compressed to give the compressed elongate sheet 127. The seton 102 may then be passed through the compressed elongate sheet 127, in a direction substantially parallel to the x-axis as shown in FIG. 9(b). As shown, a needle 901 is used to aid the passage of the seton through the compressed elongate sheet 127, however it will be appreciated that other means of forming the hole such as punching or drilling are possible. The compressed elongate sheet 127 then may be extended in opposite directions along the length of the seton 102, as shown in FIG. 9(c). Once extended, the elongate sheet 103 having multiple attachment loops 128, 129 formed by the seton 102 is provided, as shown in FIG. 9(d). The two ends of the elongate sheet may then be folded and the seton secured in a first and a second knot about each end, as previously described. In such a manner, multiple attachment loops may be created via a single operation, facilitating automated or bulk manufacture.

When folding and compressing the elongate sheet 103, it may be desirable to ensure that the creases 125, 126 or folds are not sharp. For example, the creases or folds may be formed around temporary support rollers (not shown) placed on the inside of the folds or creases 125, 126 during the folding and compression steps (a)-(c). Permanent creases in the elongate sheet 103 will thereby be avoided.

Alternately, the accordion folded elongate sheet may be formed by compressing the elongate sheet 103 between an upper former and a lower former. Typically, the upper former and the lower former are shaped with corresponding mating surfaces to allow the accordion folds to be formed when the elongate sheet 103 is clamped between the two formers. For example, the mating surfaces may be configured such that the elongate sheet adopts a shape approximating that of a sine wave extending in a direction parallel to the longitudinal axis x of the elongate sheet. Sharp creases or folds in the elongate sheet may thereby be avoided.

A longitudinal guide passage may be formed via a multitude of component passages in the two formers. The longitudinal guide passage may be configured such that when the elongate sheet 103 is clamped between the two formers, a piercing implement such as a needle or a drill may be passed though the guide passage, forming a series of holes in the elongate sheet extending along the length of the sheet. The upper and lower former may then be removed, and the seton 102 may be thread through the holes so as to form the attachment loops 128, 129. Alternatively, each of the upper and lower formers may be split into at least two sections which mate about the component passages. In such an embodiment the piercing implement may be attached to the seton 102 and thread through the elongate sheet 103 along the longitudinal guide passage. The sections of the upper and lower formers may then be separated to leave the seton 102 thread through the elongate sheet 103 multiple times so as to form the attachment loops 128, 129, e.g. as shown in FIG. 9(d). The seton 102 and the elongate sheet 103 may then be pulled taught and the seton may be secured to the seton, e.g. using knots as previously described.

The device of the invention may be used to treat simple or complex fistulas, most especially simple or complex anal or recto-vaginal fistulas. Typically, the devices of the invention may be inserted while the patient is awake, e.g. using local anaesthetic. That said, the devices may also be inserted under general anaesthetic.

For the treatment of a simple fistula, a single device according to the invention is typically used. Referring to FIG. 10(a), there is illustrated a patient with a fistula tract 131 extending from a first opening 132 located on the exterior surface of the patient's buttock or perianal skin, to a second opening 133 located inside the anal canal 134. The surgeon or care practitioner may feed the device 101 comprising the seton 102 and the elongate sheet 103 into the fistula tract 131 via the first opening 132 with the assistance of a probe 130. The probe and a first end of the seton are then manipulated through the fistula tract 131, out of the second opening 133 and out of the patient via the anal canal 134, as indicated by the arrows in FIG. 10(a). In such a manner, the elongate sheet 103 may be positioned within the fistula tract 131, with a first end of the seton extending from the second opening 133 out of the anal canal 134, and a second end of the seton extending out of the first opening 132. The probe may then be removed and the two ends of the seton tied together in a knot 135, securing the elongate sheet 103 within the fistula tract via a seton stitch as illustrated in FIG. 10(b).

As explained previously, and as is evident from FIG. 10(b), when in position the protrusions 104, 105 may act to grip or dig into the inner wall of the fistula tract 131. Accordingly, the protrusions may serve to prevent both rotational movement of the elongate sheet 103 within the fistula tract, and prevent lateral or sideways movement so as to prevent the elongate sheet 103 from sliding out of the fistula tract by rotation of the seton stitch.

Moreover, while the elongate sheet is flexible it may possess a degree of resilience. In use, to help feed the elongate sheet 103 into the fistula tract 131, the surgeon or care practitioner will typically form the sheet into a gutter or vee-shape, with the valley of the gutter or vee extending along approximately the same line as the seton, i.e. parallel to the previously defined longitudinal axis of the device. If the sheet is resilient it will have a tendency to unfold or unfurl once placed in the fistula tract, thus creating outward pressure on the protrusions into the wall of the fistula tract, thereby facilitating the securement. Moreover, by forming the elongate sheet 103 into a gutter or vee-shape with the valley of the gutter or vee extending along approximately the same line as the seton, it can be seen that in position any attempt to rotate the elongate sheet 103 about the seton will result in one of the two sets of protrusions 104, 105 digging into the internal wall of the fistula tract 131 in a direction that counters the attempted direction of rotation, irrespective of whether the elongate sheet is rotated clockwise or anticlockwise, and irrespective of whether or not the elongate sheet is resilient. Accordingly, it can be seen that the plurality of protrusions are highly effective at maintaining the tissue growth promoting matrix material in a constant position relative to the internal walls of the fistula tract. Consequently, tissue ingrowth is encouraged.

For the treatment of longer fistula tracts, or for the treatment of fistula tracts with a non-uniform internal diameter, two or more devices of the invention may be tied together end-to-end, e.g. such that one end of the elongate sheet of a first device of the invention is in close proximity or abutting one end of the elongate sheet of a second device of the invention. The elongate sheets of the first and second device according to the invention may have different widths (w), so as to correspond to the differing internal diameters of a given fistula tract.

The plurality of attachment points or loops renders the device particularly suitable for the treatment of complex fistulas. As illustrated in FIG. 11, the seton 202 of a further device 201 according to the invention may be tied to an attachment loop 136 of a first device 101 according to the invention via a knot 204. The further device comprises an elongate sheet 203 of tissue growth promoting matrix material and protrusions 205 as previously described. As will be appreciated, while the further device 201 is attached via the attachment loop 136 shown, the further device could instead be attached to any one of the additional attachment loops on the first device 101. Moreover, while not shown, additional devices of the invention may be attached to the combined tied device, either via the same attachment loop 136, via another attachment loop located on either side of the first device 101, or via an attachment loop 236 located on the further device 201. In all cases, the dimensions of the first device 101, the further device 201 and any additional devices may be selected by the end user in accordance with the fistula to be treated. Thus, it will be appreciated that almost endless variation is possible, with the surgeon or care practitioner being able to tie any number of devices together in almost any configuration so as to match the structure and degree of branching of the complex fistula to be treated.

Turning to FIG. 12, there is illustrated the insertion of the combined tied device of FIG. 11 into a complex fistula tract. Referring to FIG. 12(a), there is illustrated a patient with a complex fistula tract 137 extending from a first opening 138 located on the exterior surface of the patient's buttock or perianal skin, to a second opening 139 located inside the anal canal 141, and further extending via a branch 142 to a third opening 140 also located on the exterior surface of the patient's buttock or perianal skin.

The surgeon or care practitioner may feed the first end of the seton 102 of the first device 101 through the anal canal 141 and into the fistula tract 137 via the second opening 139 with the assistance of a first probe 143. The first probe and the first end of the seton 102 may then be manipulated through the fistula tract 137 and out of the first opening 138, as indicated by the arrows in FIG. 12(a). Similarly a first end of the seton 202 of the further device 201 which is tied to the first device may be fed through the anal canal 141 and into the fistula tract 137 via the second opening 139 with the assistance of a second probe 144. The second probe and the first end of the seton 202 may then be manipulated through the branch 142 of the fistula tract 137 and out of the third opening 140, again as indicated by the arrows in FIG. 12(a).

In such a manner, the elongate sheet 103 of the first device may be positioned within the fistula tract 137, with a first end of the seton 102 extending from the second opening 139 out of the anal canal 141, and a second end of the seton 102 extending out of the first opening 138. The further device 201 is attached to an attachment loop of the first device 101 via a knot 204 as previously discussed, and the elongate sheet 203 is positioned to extend though the length of the branch 142 of the fistula tract, such that one end of the seton 202 of the further device extends out of the third opening 140.

After the removal of any probes, the two ends of the seton 102 and the loose end of the seton 202 may then be tied together in a knot 145, securing both elongate sheets 103 and 203 within the branches of the fistula tract 137 via a seton stitch as illustrated in FIG. 12(b).

As will be appreciated, since the seton 202 of the further device is tied directly to the seton 102 of the first device, in situ the combined seton stitches and the resultant securement of the two elongate sheets 103 and 203 will be extremely robust. Moreover, the two elongate sheets 102 and 203 may further comprise a plurality of protrusions 105 and 205 in the form of serrated or castellated edges, thereby aiding securement as previously described.

The devices and the methods described may be best adapted by the surgeon or the care practitioner in view of the particular fistula to be treated. For instance, the manner of insertion of the devices of the invention is not limited to that described in relation to FIGS. 10 and 12 and other insertion techniques may readily be adopted or envisaged. For example, in relation to FIG. 12, the first device 101 and the second device 201 may be inserted into their respective branches of the fistula trach before the first device is tied to the second device via an attachment loop.

Although embodiments of the invention have been shown and described, it will be appreciated by those persons skilled in the art that the foregoing description should be regarded as a description of preferred embodiments only. The examples are not intended to limit the scope of the invention. Various modifications and embodiments can be made without departing from the scope and spirit of the invention, which is defined by the following claims only.

1. A device suitable for treating a fistula,
wherein the fistula comprises a fistula tract,
wherein the device comprises a seton and an elongate body of tissue growth promoting matrix material,
wherein the elongate body is suitable for being positioned within the fistula tract, and wherein the elongate body comprises a plurality of protrusions positioned on at least one outer surface of said elongate body.

2. The device according to claim 1, wherein each outer surface on which the plurality of protrusions are positioned is parallel or substantially parallel to the longitudinal axis of the elongate body.

3-21. (canceled)

22. The device according to claim 1, wherein:
(i) each protrusion of the plurality protrusions has a height of between 0.5 and 3 mm and has a width of between 0.5 and 5 mm; and/or
(ii) each protrusion of the plurality of protrusions is conical, triangular, triangular prismatic or pyramidal in shape.

23. The device according to claim 1, wherein the elongate body and the plurality of protrusions are of unitary construction.

24. The device according to claim 1, wherein the elongate body is an elongate sheet of tissue growth promoting matrix material, optionally wherein the plurality of protrusions are positioned on at least one outer edge surface of the elongate sheet, wherein the outer edge surface is parallel or substantially parallel to the longitudinal axis of the elongate sheet.

25. The device according to claim 24, wherein the elongate sheet comprises a first outer edge surface and a second outer edge surface,
wherein the first outer edge surface and the second outer edge surface are both parallel or substantially parallel to the longitudinal axis of the elongate sheet when laid flat,
wherein a first plurality of protrusions is formed by serrations on the first outer edge surface, and
wherein a second plurality of protrusions is formed by serrations on the second outer edge surface.

26. The device according to claim 1, wherein the tissue growth promoting matrix material comprises a scaffold, wherein the scaffold comprises a plurality of fibres, and optionally wherein:
(i) the fibres are interconnected in a non-woven disordered structure; and/or
(ii) the fibres are constructed from a biodegradable polymer such as a biodegradable polyester; and/or
(iii) the fibres are constructed from poly-L-lactic acid.

27. The device according to claim 1, wherein the seton is formed from one or more biodegradable polymers.

28. A method of manufacturing a device according to claim 1, said method comprising the step of affixing the seton to the elongate body.

29. A method of treating a fistula, the method comprising the use of a device according to claim 1.

30. A device suitable for treating a fistula,
wherein the fistula comprises a fistula tract,
wherein the device comprises a seton and an elongate body of tissue growth promoting matrix material,
wherein the elongate body is suitable for being positioned within the fistula tract, and
wherein the device comprises one or more attachment points positioned along the length of the elongate body, wherein the one or more attachment points are configured to connect the elongate body to a further seton.

31. The device according to claim 30, wherein the elongate body is an elongate sheet of tissue growth promoting matrix material.

32. The device according to claim 30, wherein each attachment point is provided in the form of an attachment loop, optionally wherein each attachment loop has an internal circumference from 5 to 20 mm, and optionally wherein the seton is thread through the elongate body of tissue growth promoting matrix material, and wherein each attachment loop is directly secured to the seton or each attachment loop is formed by the seton.

33. The device according to claim 31, wherein the elongate sheet comprises a first end and a second end, with a longitudinal axis extending from the first end to the second end,
wherein the seton extends beyond the first end of the elongate sheet, is repeatedly thread through a first side of the elongate sheet to a second side, then back though the second side of the elongate sheet to the first side, so as to form a plurality of attachment loops positioned on alternate sides along the length of the elongate sheet,
wherein the seton is optionally thread back through the first side of the elongate sheet to a second side, and
wherein the seton exits so as to extend beyond the second end of the elongate sheet,
wherein the plurality of attachment loops are configured to tie the elongate sheet to one or more further setons.

34. The device according to claim 33, wherein the seton is affixed to the elongate sheet via a first knot located proximal to the first end of the elongate sheet, and the seton is affixed to the elongate sheet via a second knot located proximal to the second end of the or elongate sheet,
optionally wherein the elongate sheet is rolled or folded at each of the first end and the second end, and the first and second knots are tied such that the loop of each knot encircles the respective rolled or folded portion of the elongate sheet.

35. The device according to claim 30, wherein the tissue growth promoting matrix material comprises a scaffold, wherein the scaffold comprises a plurality of fibres, and optionally wherein:
(i) the fibres are interconnected in a non-woven disordered structure; and/or
(ii) the fibres are constructed from a biodegradable polymer such as a biodegradable polyester; and/or
(iii) the fibres are constructed from poly-L-lactic acid.

36. The device according to claim 30, wherein the seton is formed from one or more biodegradable polymers.

37. A method of manufacturing a device according to claim 30, said method comprising the step of affixing the seton to the elongate body.

38. A method of manufacturing a device according to claim 31, said method comprising the steps of:
(i) cutting an elongate sheet of tissue growth promoting matrix material from a larger sheet of tissue growth promoting matrix material, optionally such that the elongate sheet is formed with one or more castellated or serrated outer edges that extend in a direction parallel or substantially parallel to the longitudinal axis of the elongate sheet;
(ii) folding the elongate sheet in alternate directions so as to create an accordion fold, wherein the creases of the accordion fold extend in a direction perpendicular or substantially perpendicular to the longitudinal axis of the elongate sheet;
(iii) passing the seton through the accordion folded elongate sheet, optionally with the assistance of a needle, such that the seton passes though the elongate sheet multiple times;
(iv) extending the accordion folded elongate sheet along a length of the seton; and
(v) optionally tying or otherwise securing the seton to the elongate sheet so as to prevent the elongate sheet from sliding along the seton.

39. A method of treating a fistula, the method comprising the use of a device according to claim 30.
